(19) United States
(12) Patent Application Publication
(10) Pub. No.: US 2024/0087714 A1
(43) Pub. Date: Mar. 14, 2024

(54) EXERCISE DEVICE WITH NATURAL GAIT MOTION AND COMPUTER GAMING INTERFACE

(71) Applicant: Blue Goji LLC, Austin, TX (US)

(72) Inventors: Charles Huang, Los Altos, CA (US); Christopher A. Larkin, Los Gatos, CA (US); Steven S.L. Toh, Austin, TX (US); Koley C. Porter, Austin, CA (US); Glenn F. Maynard, Austin, TX (US); Coleman F. Fung, Austin, TX (US)

(21) Appl. No.: 18/515,072

(22) Filed: Nov. 20, 2023

Related U.S. Application Data
(63) Continuation of application No. 18/463,270, filed on Sep. 7, 2023.
Application No. 18/463,270 is a continuation of application No. 18/335,070, filed on Jun. 14, 2023.
Application No. 18/335,070 is a continuation of application No. 16/990,728, filed on Aug. 11, 2020, now Pat. No. 11,756,664.
Application No. 16/990,728 is a continuation of application No. 14/012,879, filed on Aug. 28, 2013, now Pat. No. 10,737,175.
(60) Provisional application No. 61/696,068, filed on Aug. 31, 2012.

(51) Int. Cl. G16H 20/30; A63B 24/00; A63F 13/428

(52) U.S. Cl. CPC G16H 20/30; A63B 24/0003; A63F 13/428; A63F 13/211

(57) ABSTRACT

An exercise machine with a more natural gait and computer gaming interface which, in some embodiments, further allows for a variable stride length while in use. In one configuration, each side of the exercise machine has a first crank assembly connected at an upper location on a pedal arm and a second crank connected at a middle location on the pedal arm, and a pedal is attached to a lower location of the pedal arm. The first crank assembly is connected to the pedal arm by a crank arm. The second crank assembly is connected to the pedal arm via a crank arm pivotally attached to a crank link. The path of each pedal is determined by the lengths of crank arms and crank links of the first and second crank assemblies rotating in synchronous motion.

### CROSS-REFERENCE TO RELATED APPLICATIONS

Priority is claimed in the application data sheet to the following patents or patent applications, each of which is expressly incorporated herein by reference in its entirety:
Ser. No. 18/463,270
Ser. No. 18/335,070
Ser. No. 16/990,728
Ser. No. 14/012,879
61/696,068

### BACKGROUND OF THE INVENTION

### Field of the Art

The present invention generally relates to exercise equipment, and more particularly, to exercise devices providing walking or running motions.

### Discussion of the State of the Art

The need for physical exercise is especially important in today's world of drive through windows and sitting in front of a computer all day. It is suggested that cardiovascular exercise is most beneficial if the user can obtain an elevated heart rate for at least twenty minutes of sustained exercise. This type of exercise may vary according to the needs and personal preference of the user, but for many people a movement that employs larger muscles, such as those which move the legs and hips may be desirable, as these are capable of doing the most work. For many sedentary individuals the accumulation of excess bodyfat may be a concern. The more work the body does, the more energy the body uses to perform that work. In the human body, most energy storage is in the form of bodyfat. Therefore, using larger muscles to do more work may enable a quicker end result on reducing bodyfat and increasing cardiovascular health. Adding an upper body system to an exercise device may further help by taxing additional muscle groups and increase the work output of the body over time.

It may be beneficial for the user to perform an activity that has a familiar basis to the normal function of the body, such as walking or running. Walking may be preferred for many users, as the impact forces on the legs are less compared to those found in running for the same person, even at the same speed. Using a walking motion may enable some participants to perform the activity that they could not perform under the higher running loads. Also, the lower impact stress may allow some people to perform the activity longer and therefore have a higher cumulative energy expenditure compared to running, even considering the higher energy expenditure per unit of time with running as compared to walking. Increasing the energy expenditure and still maintaining a walking gait may be accomplished by increasing the force required to move the pedals of a machine that simulates walking, or altering the angle to simulate walking up a hill. Either or both may provide a useful alternative to walking on a street or road where the user may be subjected to extreme weather conditions, traffic or physical dangers not found in their home or other controlled environment.

Treadmills have typically been used, but they can be large, expensive and noisy. The noise is due at least partially to the friction between the moving belt and the supporting deck under the belt that occurs with each step. Where there is friction there is wear. The decks must be regularly replaced or lubricated and the belts replaced.

It should, therefore, be appreciated that there is a need for an exercise device that allows the user to simulate walking or other bipedal movement that can be done indoors, with a small footprint and with minimal noise and parts wear. The present invention fulfills this need and others.

Existing elliptical exercise machines attempt to provide a low-impact exercise motion, but force a gait that is strictly elliptical in nature, which is not consistent with a natural walking motion. Further, existing elliptical exercise machines do not allow for a variable stride length. What is needed is a low-impact exercise machine similar to existing elliptical-type exercise machines, but with a more natural gait motion.

Further, as exercise can be repetitive and boring for some people, an exercise device is needed which encourages physical activity through gaming.

### SUMMARY OF THE INVENTION

Accordingly, the inventor has conceived and reduced to practice an exercise machine with a more natural gait and computer gaming interface which, in some embodiments, further allows for a variable stride length while in use. In one configuration, each side of the exercise machine has a first crank assembly connected at an upper location on a pedal arm and a second crank connected at a middle location on the pedal arm, and a pedal is attached to a lower location of the pedal arm. The first crank assembly is connected to the pedal arm by a crank arm. The second crank assembly is connected to the pedal arm via a crank arm pivotally attached to a crank link. The path of each pedal is determined by the lengths of crank arms and crank links of the first and second crank assemblies rotating in synchronous motion.

According to a preferred embodiment, an exercise device and computer game controller is disclosed, comprising: a frame; a first and a second crank system mounted opposing sides of the frame adapted for positioning a user therebetween, each of the first and second crank system comprising a first crank assembly rotatable about a first axis and a second crank assembly rotatable about a second axis with the first and second axes each being displaced from the other, each of the first crank assemblies comprising a first crank arm, and each of the second crank assemblies comprising a second crank arm; a pair of synchronous control system assemblies one mechanically coupled to the first crank system and the other mechanically coupled to the second crank system, each synchronous control system assembly of the pair of synchronous control system assemblies being mechanically coupled to the first and second crank assemblies of the corresponding crank system in a configuration that allows for 360 degrees of synchronous rotation of the first and second crank assemblies, wherein each first crank arm of each first crank assembly rotates in the same direction as the second crank arm of each second crank assembly by the corresponding synchronous control system assembly of the pair of synchronous control system assemblies; a pair of pedal arms, one coupled to the first crank system and the other coupled to the second crank system, each of the pair of pedal arms being pivotally coupled at a first end to the first crank arm of the first crank assembly and being pivotally coupled at a second end to a pedal; a pair of movable crank links, one corresponding to each of the first and second crank systems, each of the pair of movable crank links comprising a first attachment point, a second attachment point, and a flexible attachment between the first attachment point and second attachment point, wherein the first attachment point of each movable crank link is pivotally coupled to the pedal arm of its corresponding crank system and the second attachment point of each movable crank is pivotally coupled to the second crank arm of its corresponding second crank assembly; and a sensor configured to measure a movement of a component of the exercise device and transmit the measurement as data to a compatible computing device.

According to an aspect of an embodiment, the system of claim 1, wherein the sensor is configured to continually or periodically measure a distance between the first attachment point and second attachment point.

According to an aspect of an embodiment, the sensor is configured to calculate a stride length and stride period based on the continually or periodically measured distance.

According to an aspect of an embodiment, the sensor is configured to measure a speed of operation of one of the pair of synchronous control systems.

According to an aspect of an embodiment, the sensor is configured to calculate a power output of the user based on the measured speed of operation.

According to an aspect of an embodiment, a game controller is affixed to the exercise device and configured to transmit the game control data to the compatible computing device.

According to an aspect of an embodiment, aa pair of game controllers are affixed to the exercise device and configured to transmit the game control data to the compatible computing device, wherein each game controller is affixed to opposite sides of the exercise device and is accessible to the hands of the user.

### BRIEF DESCRIPTION OF THE DRAWING FIGURES

Embodiments of the present invention will now be described, by way of example only, with reference to the following drawings, in which:

FIG. 1 is an isometric view of an exercise device, presented in accordance with the present invention.

FIG. 2 is an isometric view of an exercise device which includes drive handles, the Figure presented in accordance with the present invention.

FIG. 3 is an isometric view of the exercise device shown as shown in FIG. 2, shown without a simulated user and from a right front viewpoint.

FIG. 4 is an isometric view from the left rear of the exercise device as shown in FIG. 3.

FIG. 5 is an isometric view from the right rear of the exercise device as shown in FIG. 3 with a portion of the frame removed to more clearly show detail of the system.

FIG. 6 is a side view of the exercise device as shown in FIG. 3 and illustrating a tracing of the movement of the pedals.

FIG. 7 is a side view of a modified version of the exercise device shown in FIG. 3, wherein the upper frame may be movably mounted to the base frame, a tracing of the pedal path at this inclined position is shown in contrast to the flat position of the upper frame in FIG. 6.

FIG. 8 is an isometric view from the front left of the exercise device shown in FIG. 7.

FIG. 9 is an isometric view from the right rear of the exercise device shown in FIG. 7.

FIG. 10 is an isometric view from the left front of the exercise device as shown in FIG. 1 but where the rigid crank links have been replaced with movable crank links.

FIG. 11 is an isometric view from the left rear of the exercise device as shown FIG. 2, but where the rigid crank links have been replaced with movable crank links.

FIG. 12 is an isometric view of a movable crank link removed from the rest of the exercise device as shown in FIG. 11.

FIG. 13 is a front view of the exercise device as shown in FIG. 11.

FIG. 14 is a side view of the exercise device as shown in FIG. 11 illustrating the tracing of the pedal paths of the movable crank links in a neutral position and also as may be seen when the movable crank links are moved by the user.

FIG. 15 is an isometric view from the left rear of an exercise device produced in accordance with the present invention in which the crank link pin is movably mounted on the pedal arm.

FIG. 16 is a detailed view of the crank link pin and pedal arm as shown in FIG. 15, the view cut along the line 16-16 in FIG. 15.

FIG. 17 is a side view of the exercise device as shown in FIG. 15 including the tracing of the pedals as altered by the movement of the crank link pin on the pedal arm.

FIG. 18 is an isometric view of an alternative embodiment of the exercise device as shown in FIG. 1.

FIG. 19 is a detail cut away of the support frame and transmission of the exercise device of FIG. 18, cut along line 19-19.

FIG. 20 is an isometric view of the exercise device of FIG. 18, shown from a front, side perspective with a portion of the base frame removed.

FIG. 21 is a side view of the exercise device of FIG. 18 shown in a non-inclined orientation.

FIG. 22 is a side view of the exercise device of FIG. 21, shown in an inclined orientation.

FIG. 23 is an isometric view of a portion of the exercise device of FIG. 22, shown from a front, side perspective with a section of the base frame removed.

FIG. 24 is a diagram illustrating an exemplary sensor for detecting movement of a movable crank link.

FIG. 25 is a diagram illustrating another exemplary sensor for detecting movement of a movable crank link.

FIG. 26 is a diagram illustrating an exemplary method for calculation of stride length in an embodiment having movable crank links.

FIG. 27 is a diagram illustrating use of a direct current generator to determine power output of a user during exercise.

FIG. 28 is a diagram illustrating an exemplary footpad for detecting pressure and lift of a user's foot during exercise.

FIG. 29 is a side elevation view of a movable crank link utilizing leaf springs for variable stride length resistance.

FIG. 30 is a side elevation view of a movable crank link utilizing elastomeric bands for variable stride length resistance.

FIG. 31 is a side elevation view of a single-piece movable crank link utilizing a single leaf spring for variable stride length resistance.

FIG. 32 is a diagram illustrating an embodiment of an exercise device having a single synchronized control system.

FIG. 33 is a diagram illustrating another embodiment of an exercise device having a single synchronized control system.

FIG. 34 is a block diagram illustrating components of the adaptable fitness system, according to one embodiment.

FIG. 35 is a block diagram illustrating the components of the motion sensing device, according to one embodiment.

FIG. 36 illustrates a motion sensing device, according to one embodiment.

FIG. 37 illustrates a hand controller, according to one embodiment.

FIG. 38 illustrates an example of hand controllers mounted to handlebars of a fitness device, according to one embodiment.

FIG. 39 is a flowchart illustrating an example process for interacting with a fitness application based on the user's interaction with the fitness system, according to one embodiment.

FIG. 40 illustrates an example process of a user manipulating events in a fitness application based on the user's interaction with the fitness system in real time, according to one embodiment.

FIG. 41 is a flow chart illustrating an example process for determining a step taken by the user, based on motion data, according to one embodiment

FIG. 42 is a flow chart illustrating an example process for interacting with the adaptable fitness system, according to one embodiment.

FIG. 43 illustrates an exemplary computing environment on which an embodiment described herein may be implemented, in full or in part.

### DETAILED DESCRIPTION OF THE INVENTION

The inventor has conceived and reduce to practice an exercise machine with a more natural gait and computer gaming interface which, in some embodiments, further allows for a variable stride length while in use. In one configuration, each side of the exercise machine has a first crank assembly connected at an upper location on a pedal arm and a second crank connected at a middle location on the pedal arm, and a pedal is attached to a lower location of the pedal arm. The first crank assembly is connected to the pedal arm by a crank arm. The second crank assembly is connected to the pedal arm via a crank arm pivotally attached to a crank link. The path of each pedal is determined by the lengths of crank arms and crank links of the first and second crank assemblies rotating in synchronous motion.

A control system may be provided that may be in communication with the first crank assembly and the second crank assembly, the control system may provide a synchronous movement of the first crank assembly relative to the second crank assembly. A pedal arm may have a first end pivotally coupled to the first crank assembly and a pedal positioned on a second end of the pedal arm. In addition, a crank link may have one end coupled to the second crank assembly and a second end coupled to the pedal arm. The first crank link may be movably coupled to a location on the first pedal arm and then moved to a second position on the first pedal arm, thereby changing the path of movement of the pedals.

The control system may include a drive shaft rotatably connected to the frame, which may provide mechanical communication between the first crank arm and the second crank arm of the first crank assembly. The control system may also include a drive member selected from the groups consisting of a belt, roller chain, a synchronous belt, a v-belt and a poly-v belt. The control system may also include a torque linkage including a link rod rotatably coupled to the second crank assembly and the drive shaft. The torque linkage may transfer power between the second crank assembly and the drive shaft operating in cooperation with the drive member. A braking system may be provided that may be in mechanical communication with the drive shaft, which may provide a resistance to movement of first crank arm and the second crank arm of the first crank assembly and the second crank assembly.

The exercise device may also include a support frame which may support the first crank assembly and the second crank assembly. The support frame may be movably mounted to a base frame, such that the orientation of the first axis or the second axis may be altered with respect to the base frame and thereby vary the path of the pedals.

The system may include a pair of drive handles pivotally coupled to the frame, each one of the pair of drive handles may include a hand grip on a first end and a drive lever on a second end. A handle link may also be provided with a first end pivotally coupled to the first crank assembly and a second end pivotally coupled to the drive lever.

For purposes of summarizing the invention and the advantages achieved over the prior art, certain advantages of the invention have been described herein. Of course, it is to be understood that not necessarily all such advantages can be achieved in accordance with any particular embodiment of the invention. Thus, for example, those skilled in the art will recognize that the invention may be embodied or carried out in a manner that achieves or optimizes one advantage or group of advantages as taught herein without necessarily achieving other advantages as may be taught or suggested herein.

All of these embodiments are intended to be within the scope of the invention herein disclosed. These and other embodiments of the present invention will become readily apparent to those skilled in the art from the following description of the preferred embodiments and drawings, the invention not being limited to any particular preferred embodiment(s) disclosed.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### DETAILED DESCRIPTIONS OF THE DRAWING FIGURES

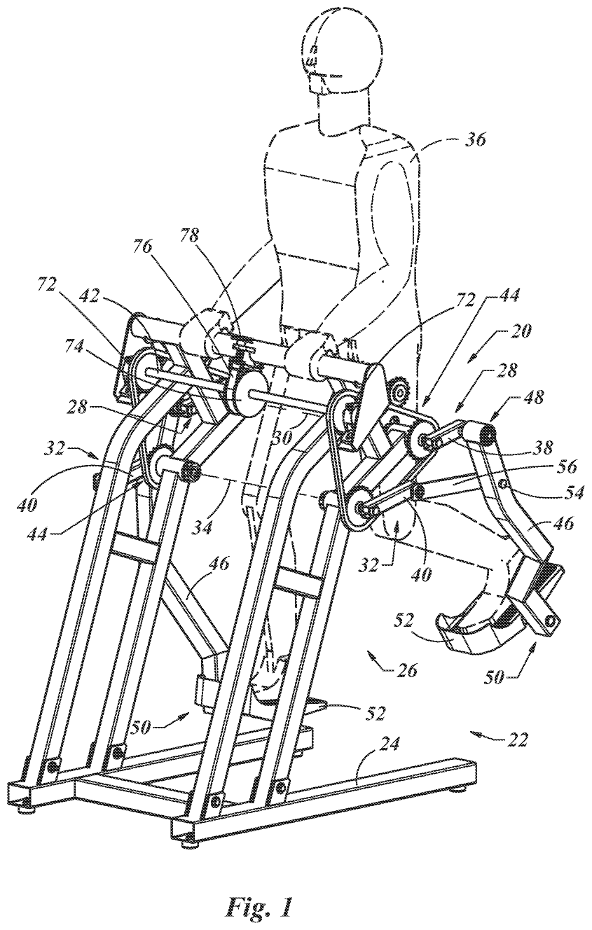

With reference to the illustrative drawings, and particularly to FIG. 1, there is shown an exercise device 20. The exercise device 20 may include a frame 22, which may include a base frame 24 and a support frame 26. The support frame 26 may be adapted to support a first crank assembly 28, which may be journaled to the support frame 26 to allow for rotational movement of the first crank assembly 28 about a first axis 30. A second crank assembly 32 may be journaled to the support frame 26 about a second axis 34. In this disclosure the locations of the first axis 30 and the second axis 34 may be positioned to cause a potential interference with a user 36 using the exercise device 20. To solve this problem, the first crank assembly 28 and the second crank assembly 32 may each be split into a left crankset and a right crankset. To maintain that the first crank arms 38 and the second crank arms 40 of the first crank assembly 28 and the second crank assembly 32 respectively, may be positioned to be 180° out of phase with the other crank arm (38 and 40) of each crankset (28 and 32), a drive shaft 42 may also be journaled to the support frame 26 as illustrated.

It may be desirable for the first crank assembly 28 and the second crank assembly 32 to move in a synchronous manner with respect to one another. This may be accomplished by the use of a control system 44. The control system 44 may be comprised of a roller chain, a drive belt, a gearing system or any other mechanical transmission elements known in the art. In that the first axis 30 and the second axis 34 may not allow a direct communication between the right and left portions of the first crank assembly 28 or the second crank assembly 32, the control system 44 may also be in mechanical communication with the driveshaft 42. In that the driveshaft 42 may be located in position to mechanically span the entire width of the exercise device 20, the driveshaft 42 with the control system 44 may then act to connect the left and right portions of each of the first crank assembly 28 and the second crank assembly 32 as well as provide for synchronous rotation of the first crank assembly 28 with respect to the second crank assembly 32.

A pedal arm 46 may be rotatably coupled to the first crank assembly 28 on a first end 48 of the pedal arm 46. On a second end 50 of the pedal arm 46 a pedal 52 may be provided, which may be adapted to support the weight of the user 36. Between the first end 48 and the second end 50 of the pedal arm 46, a crank link pin 54 may be provided. The crank link pin 54 may be pivotally coupled to a crank link 56 and the crank link 56 may also be coupled to the second crank assembly 32, thereby connecting the pedal arm 46 to the second crank assembly 32.

It is important to note that in these preferred embodiments of the exercise device 20, the drive shaft 42 is positioned such that it is not in line with the first axis 30 or the second axis 34. That is because both the first axis 30 and the second axis 34 may be positioned to potentially interfere with the movement of the user 36. As such, the drive shaft 42 may be moved to a position that is unlikely to interfere with the movement of the legs of the user 36. It is possible that the second axis 34, and the second crank assembly 32, could be moved far enough forward to avoid the legs of the user 36. If that were done, the drive shaft 42 could directly connect the second crank assembly 32. The crank link 56 may then be increased in length in accordance with the increase in dimension of the relocation of the second axis 34 relative to the current position. This would eliminate the need for some components in the system. The applicant recognizes this could be done and hereby includes this as a variation to the disclosed embodiments even though this is not shown in any of the Figures.

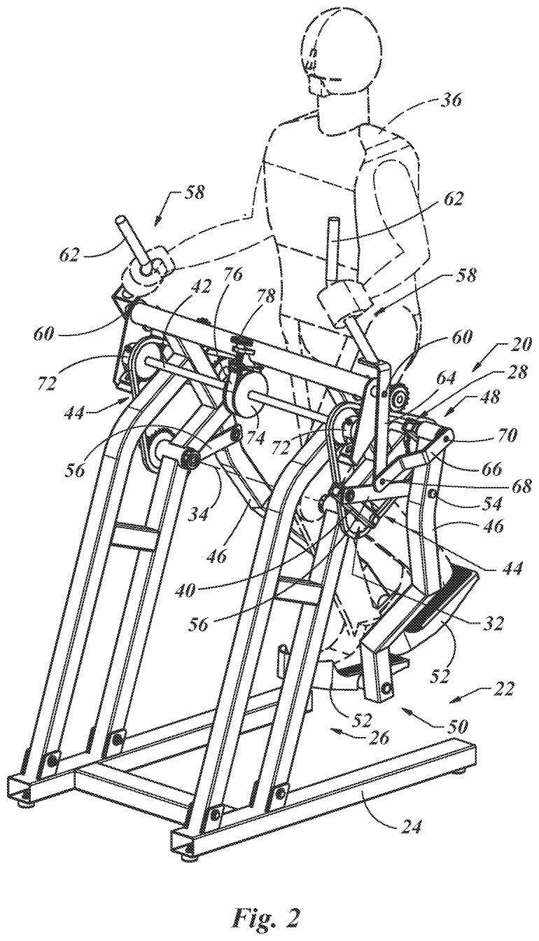

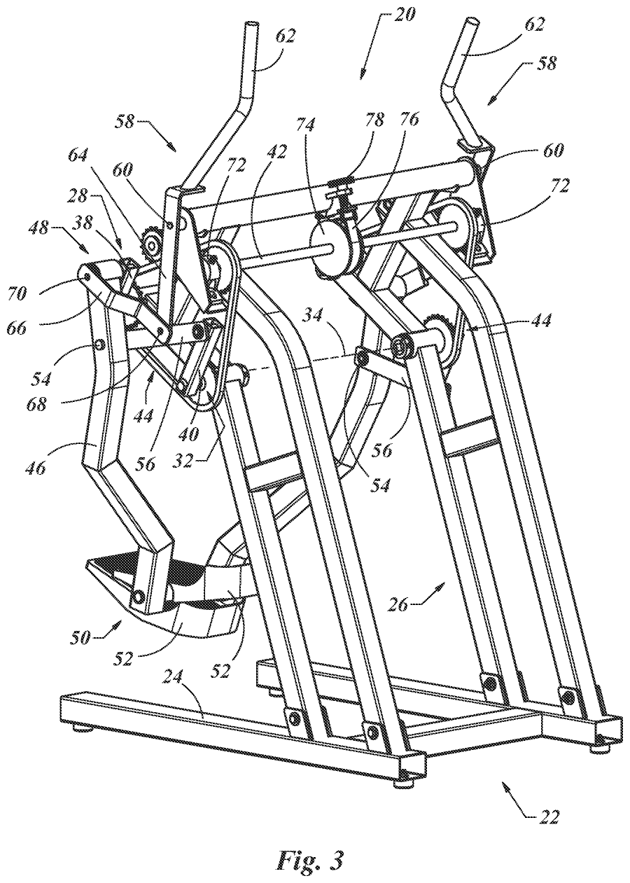

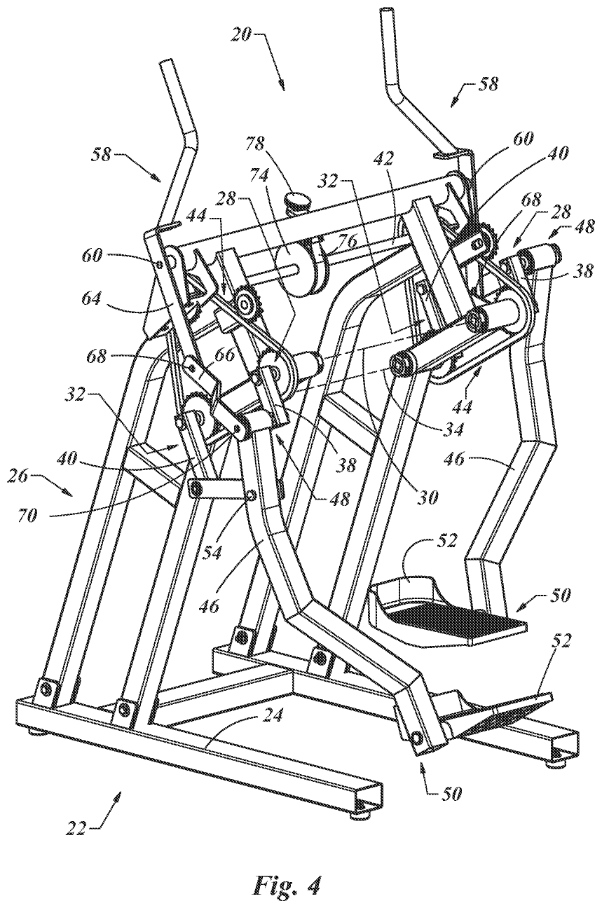

With reference to FIGS. 2-5, a similar exercise device 20 is shown with the addition of drive handles 58, which may be pivotally coupled to the frame 22 about a pivot pin 60. The drive handles 58 may each include a hand grip 62 adapted to be gripped by the hands of the user 36. The drive handles 58 may also include a drive lever 64, which may be coupled to a handle link 66 about a pivot 68. The handle link 66 may include a second pivot 70, which may be mounted to the first crank assembly 28 at second pivot 70. Therefore, the user 36 may be positioned on the pedals 52 of the pedal arms 46 and perform a walking motion such that the first crank assembly 28 may provide a vertical displacement of each pedal arm 46, and therefore the pedal 52, while the second crank assembly 32 may provide for horizontal displacement of the pedal 52 by way of the crank link 56. This movement may drive the driveshaft 42, thus causing it to rotate about the bearings 72, which may be mounted to the support frame 26 of the frame 22.

To provide a smooth movement of the pedals 52 a flywheel 74 may be coupled to the driveshaft 42, whereby rotation of the driveshaft 42 about the bearings 72 may cause a similar rotation of the flywheel 74. It may also be desirable to include a braking system to resist the movement of the pedals 52. A braking system may include a friction strap 76 mounted to a screw 78, which may be mounted to the support frame 26. Therefore, as the screw 78 is advanced this may increase the tension in the friction strap 76, which may increase the drag and therefore increase the resistance to movement of the flywheel 74. This may increase the work necessary to be provided by the user 36, thus increasing the intensity of the exercise. In a similar manner, any magnetic form of resistance known to the art, such as an electric motor, an eddy current brake or any other braking system may be used in place of the combination of the friction strap 76 and screw 78.

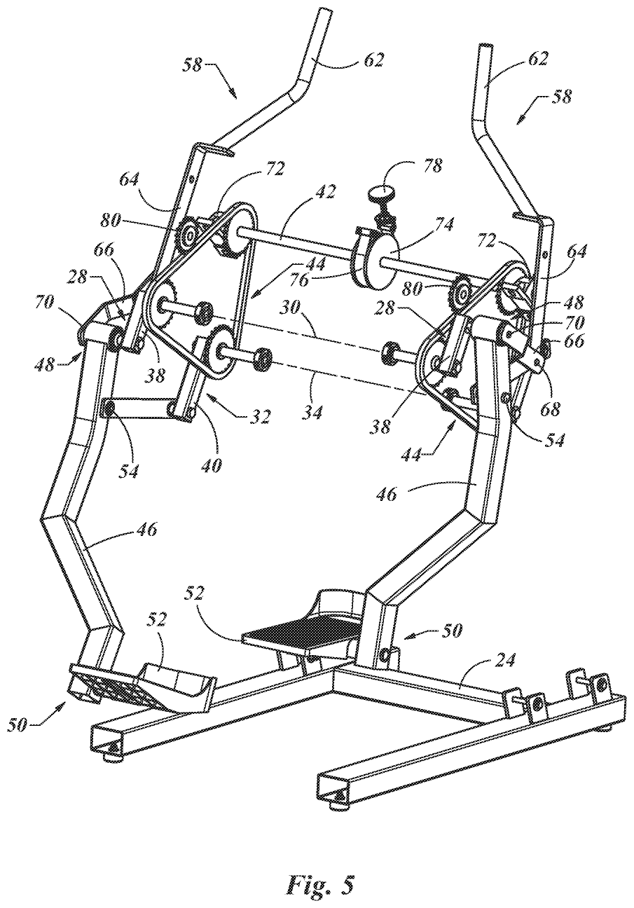

With particular attention to FIG. 5, the support frame 26 has been removed to better illustrate this embodiment of the present invention 20. The elements as previously shown and described are consistent with that as presented above. What may also be more clearly seen in this Figure is the potential use of a tension device to optimize the function of the control system 44. As noted, the control system 44 may include a roller chain or drive belt. It may be desirable to use an idler pulley 80 positioned relative to the drive belt or roller chain of the control system 44 so as to displace a portion of the roller chain or drive belt, thus creating proper tension in the drive belt or roller chain to optimize the function of the control system 44.

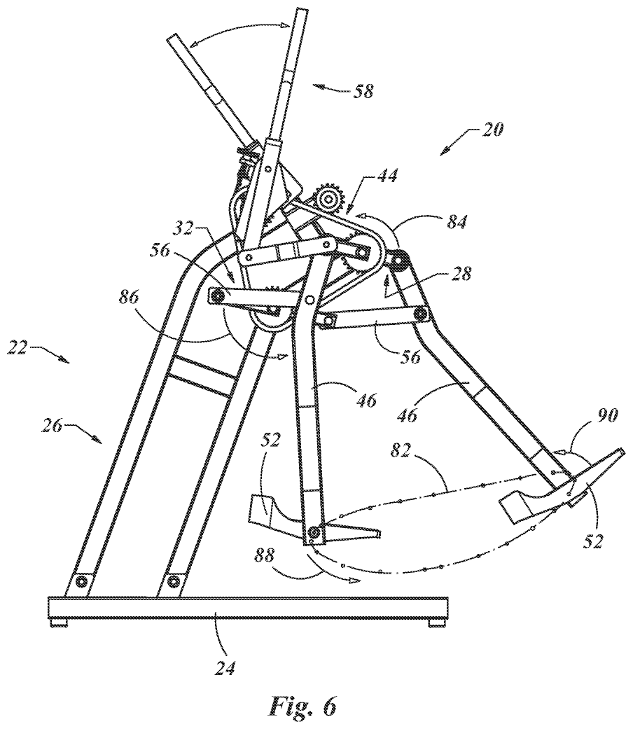

With regard to FIG. 6, a side view of the exercise device 20 is shown with a trace of a pedal path 82. In this view it can be seen how the pedal arms 46 may be connected to the first crank assembly 28. As the first crank assembly 28 rotates in the direction of the drive arrow 84 the pedal arm 46 on the left may move downward while the pedal arm 46 on the right may move upward. The control system 44 may provide the same direction of rotation of the second crank assembly 32, as designated by the second arrow 86. As the second crank assembly 32 rotates in a counter clockwise direction (in this view), the second crank assembly 32 may drive the crank link 56 to alter the angular orientation of the pedal arms 46, as they are driven by the first crank assembly 28. In this view and in this relative position of the pedal arms 46, the pedal arm 46 on the left may move generally down and to the right, as shown by the first pedal arrow 88, while the pedal arm 46 on the right may move generally up and to the right until it changes direction to move to the left and moving down as illustrated by the second pedal arrow 90. An example of a complete path of the pedals 52 may be shown moving along the trace of the pedal path 82.

It may be desirable to change the resistance of the exercise device 20. Any number of users may have different physical capabilities and therefore it may be desirable to have the exercise device conform to those varying capabilities. In addition, as a person uses an exercise device, it is likely the person's physical fitness level will improve. As such, in order to continue to make physiological gains from the exercise, it may be desirable for the exercise device to increase in its ability to stress the body of the user by increasing the workload.

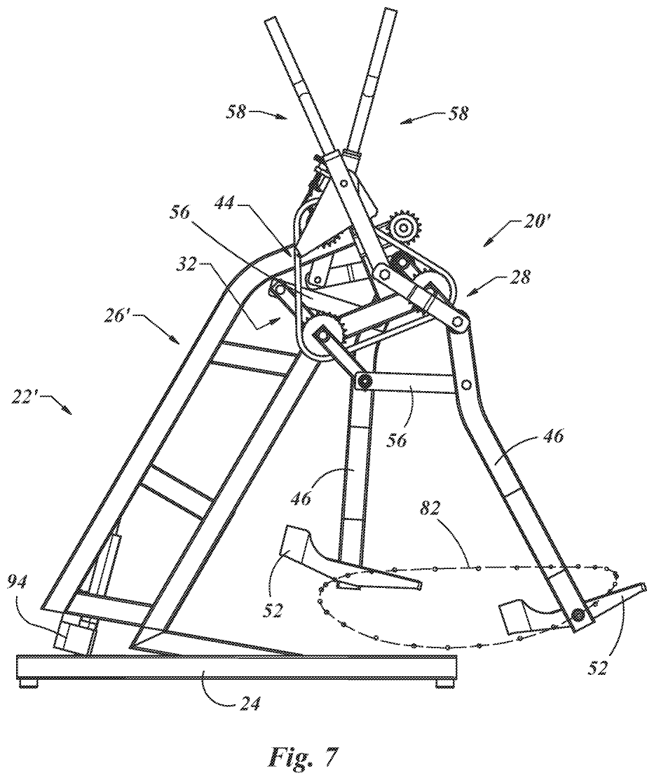

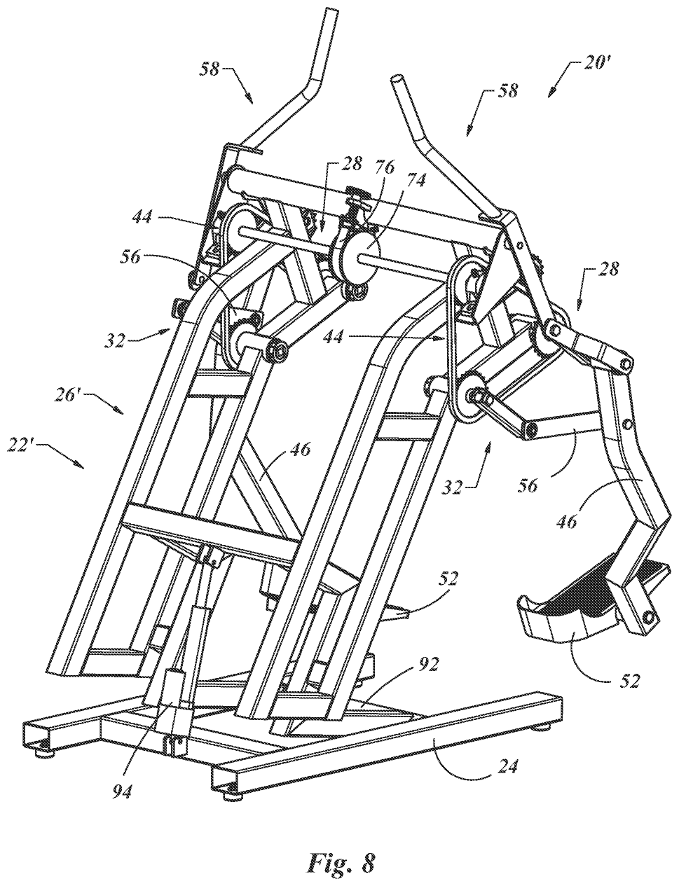

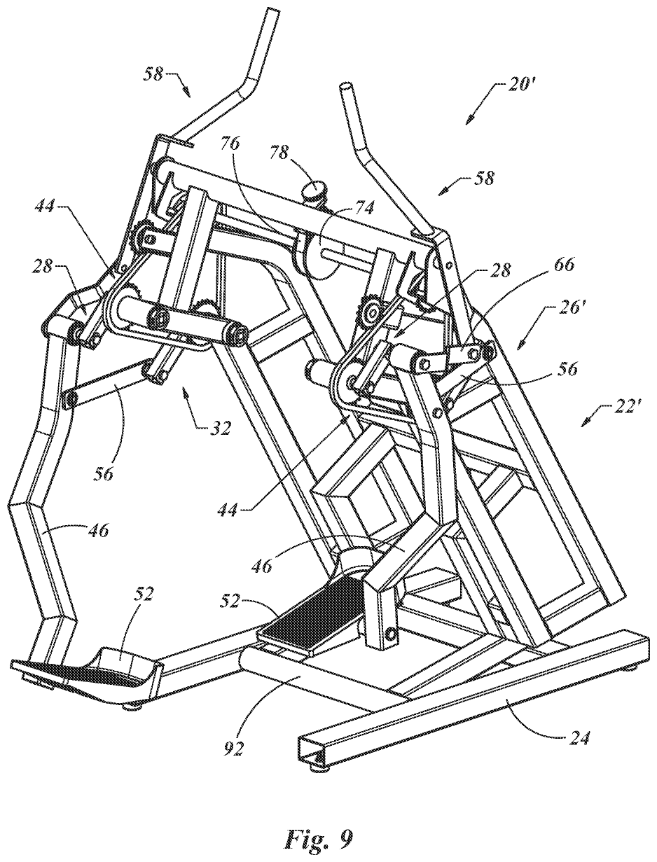

As noted, one method to increase the workload in the present exercise device 20 is by providing resistance to rotation of the flywheel 74 by way of the friction strap 76 or any other form of resistance, such as any number of electromagnetic braking systems. An alternative may be to increase the angle of the pedal path. A solution to do so is illustrated in FIGS. 7-9. As was shown in FIG. 6, the trace of the pedal path 82 may be similar to that of a normal walking gait of a person. This walking gait may be similar to that of a person walking on a treadmill with no incline. To increase the caloric expenditure the user may increase the incline or grade of the treadmill deck. According to the American College of Sports Medicine, a person walking on a flat 0% grade versus walking the same speed at a 10% grade will have an estimated metabolic requirement of over 2.3 times greater walking at the inclined grade. Therefore, changing the angle of incline of the gait pattern of the user may also increase energy expenditure of the user during the exercise session. In that a walking gait may result in significantly lower impact stress on the joints of the user, it may be desirable for a user to exercise in this manner as opposed to running and expend a similar amount of energy per unit of time performing the inclined walking exercise.

With regard to FIGS. 7-9, an alternative version of the exercise device 20′ is shown which may incorporate the ability to change the angle of the pedal path of the user. The frame 22′ may include the base frame 24, which may be supported on the ground or floor as with the other embodiments. The support frame 26′ may include a support frame pivot 92, which may be coupled to the base frame 24. This may allow the support frame 26′ to change its angular orientation with respect to the base frame 24 by pivoting about the support frame pivot 92. An actuator 94 may be positioned between the base frame 24 and the support frame 26′ wherein as the actuator 94 extends, the angle between the support frame 26′ and the base frame 24 may be increased. As may be illustrated in FIG. 7, the trace of the pedal path 82 may have the same overall shape as that shown in FIG. 6 where the exercise device 20 is at a standard flat position, but the trace of the pedal path 82 may be rotated in FIG. 7 in accordance with the increase in angle of the support frame 26′ relative to the base frame 24. This may allow a user to increase the workload of the exercise session to a varying degree according to the amount of incline the user selects between the support frame 26′ and the base frame 24.

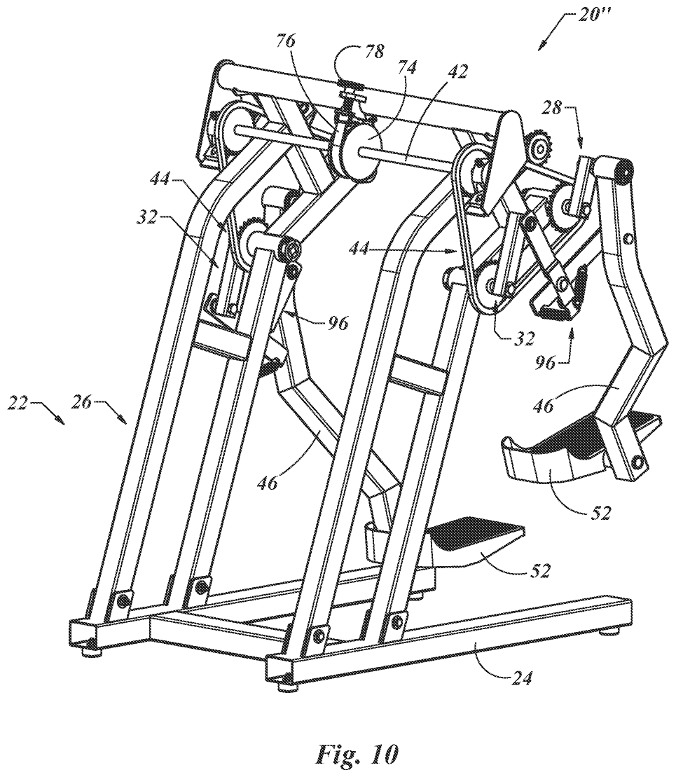

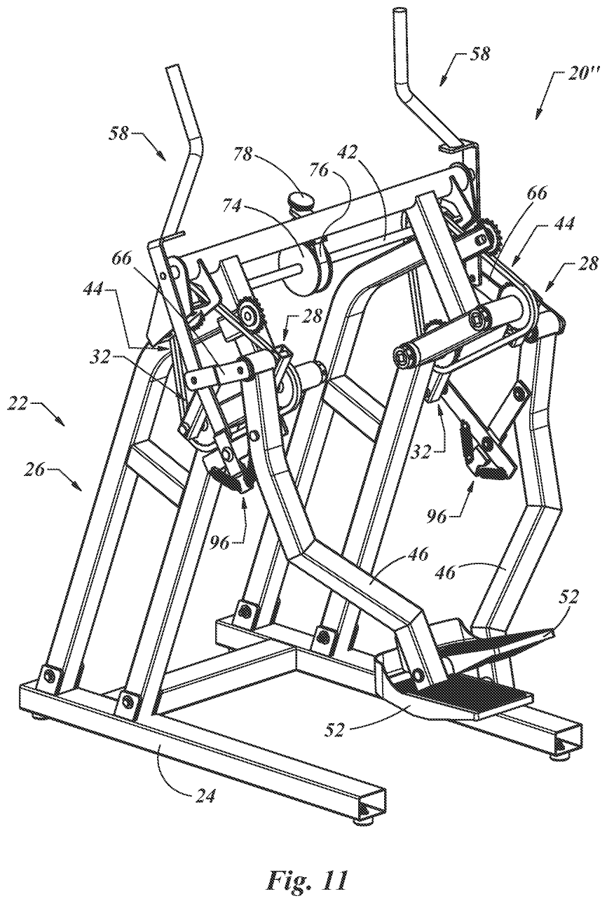

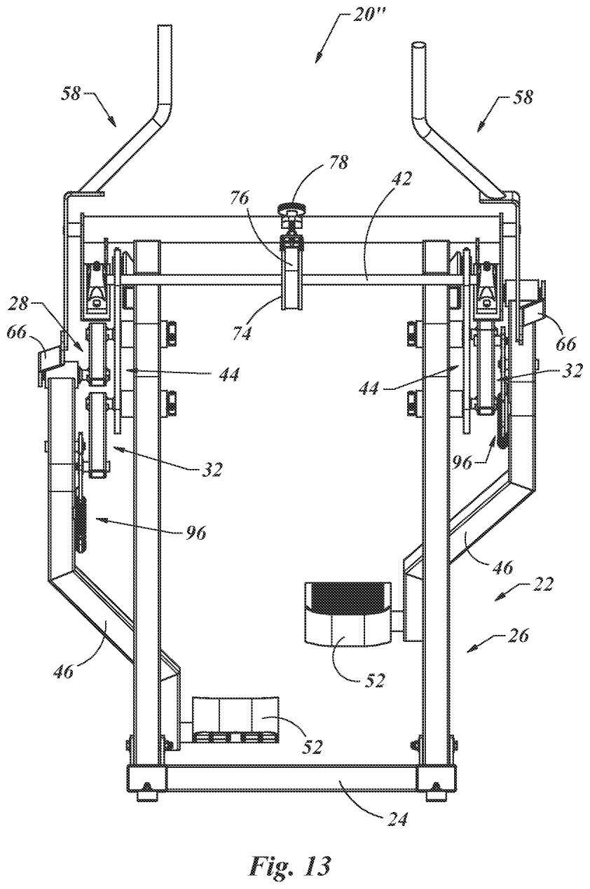

It may be desirable to enable the user to change the stride length of the pedal path. An example of how this may be accomplished is shown in FIGS. 10-14. The general function of the exercise device 20″ may be the same as previously shown and described with the exception of the crank link 56 may be replaced with a movable crank link 96. The movable crank link 96 may include a first link 98 and a second link 100, which may be pivotally coupled about a crank link pin 102. A first spring 104 may be coupled to the first link 98 and the second link 100 and in a similar but opposite manner, a second spring 106 may likewise be coupled to the first link 98 and the second link 100. The orientation of the first spring 104 and the second spring 106 may apply a balanced couple to the first link 98 relative to the second link 100 so that the distance “x” between the bearing attachments 108 may be biased to a predetermined dimension. Though the movable crank link 96 may be biased to a dimension “x” between the bearing attachments 108, if a force is applied to the movable crank link 96 in a direction to decrease the value of “x”, the first spring 104 may increase in length and the second spring 106 may decrease in length.

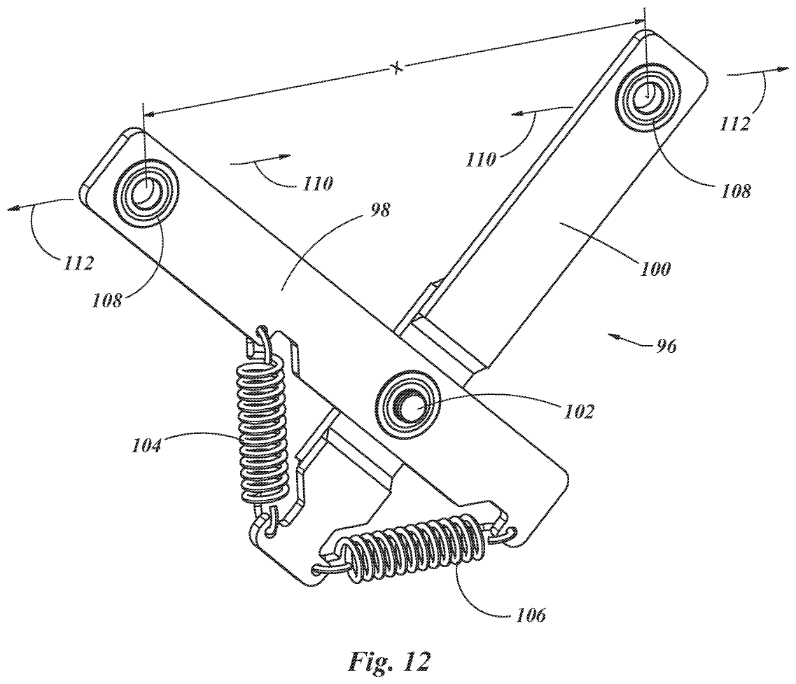

A spring may obey “Hook's Law” in that the force needed to deform a spring by some distance is proportional to that distance. Therefore in this example, if a force is applied to cause the distance “x” to decrease, as depicted by the compression arrows 110 shown in FIG. 12, the length of the first spring 104 may increase and therefore the force applied by the spring to the first link 98 and the second link 100 would also proportionally increase. The length of the second spring 106 may decrease, and therefore the force applied to the first link 98 and the second link 100 by the second spring 106 may proportionally decrease. The opposite may be found in that as force is applied to the movable crank link 96 that results in increasing the dimension “x”, as depicted by the extension arrows 112, the first spring 104 may decrease in length (and applied force) and the second spring 106 may increase in length (and applied force).

The result is the movable crank link 96 may allow for the dimension “x” to be increased or decreased by forces applied to the movable crank link 96, but the action of the first spring 104 and the second spring 106 may provide a pair of forces that may be balanced optimally when the dimension “x” is at a predetermined value. This combination may allow for displacement of the first link 98 relative to the second link 100 of the movable crank link 96 to allow the value of “x” to vary, but yet provide a bias to return the orientation of the movable crank link 96 to that so the dimension “x” may be a predetermined value.

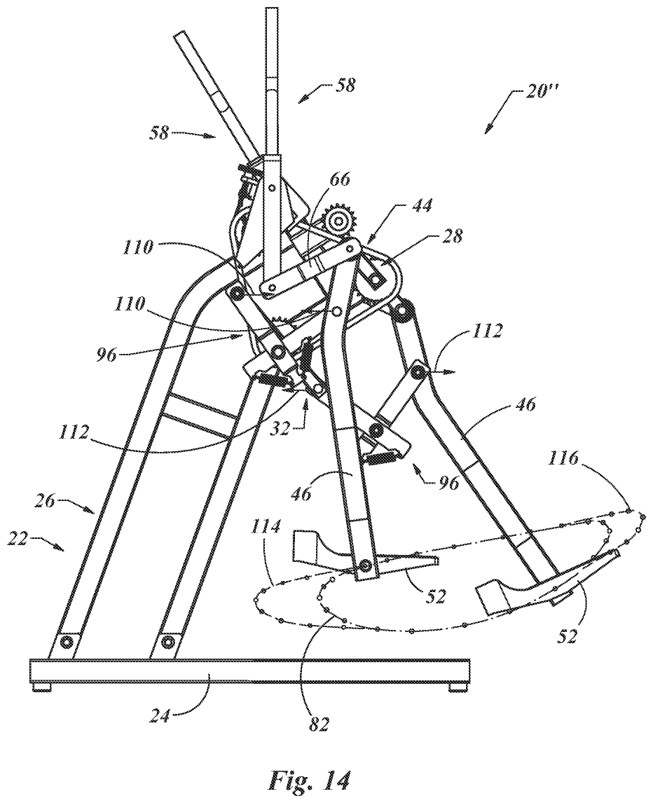

When used on the exercise device 20″, the force applied to the movable crank links 96 may be applied by the user to the pedals 52. This may be illustrated in more detail in FIG. 14, where the previously illustrated trace of the pedal path 82 is presented as it may be generated if the movable crank links 96 maintain a set angular orientation of the first link 98 relative to the second link 100, so as to provide a constant dimension “x”, as shown in FIG. 12. If the user provides a force to the forward pedal 52 (left one pedal in FIG. 14) to extend it forward beyond the trace of the pedal path 82, a force may be applied to the movable crank link 96 coupled to the forward pedal arm 46 (left one in FIG. 14) to provide a force to move the movable crank link 96 in the direction of the compression arrows 110, or to reduce the value of dimension “x”. This may allow the pedal 52 supporting the front foot to extend beyond the trace of the pedal path 82 on the forward pedal 52 (left in FIG. 14) to generate a front pedal path trace extension 114.

In a similar but opposite manner, if the user wishes to extend the pedal 52 supporting the back foot farther back than what may be illustrated by the trace of the pedal path 82, the user may apply a force to the rear pedal 52, (right one in FIG. 14) which may cause the movable crank link 96 connected to the pedal arm 46 supporting the rear pedal 52 (right one in FIG. 14) to move in the direction of the extension arrows 112, or to increase the value of dimension “x”. This may allow the pedal 52 supporting the rear foot to extend beyond the trace of the pedal path 82 on the rear pedal 52 (right one in FIG. 14) to generate a rear pedal path trace extension 116. The combination may provide an increased stride length of the pedals 52, which may accommodate a taller person, or anyone else that may want a longer stride length. This may be accomplished by the user overcoming the resistance provided by the first spring 104 and the second spring 106 as needed and to the extent desired by the user.

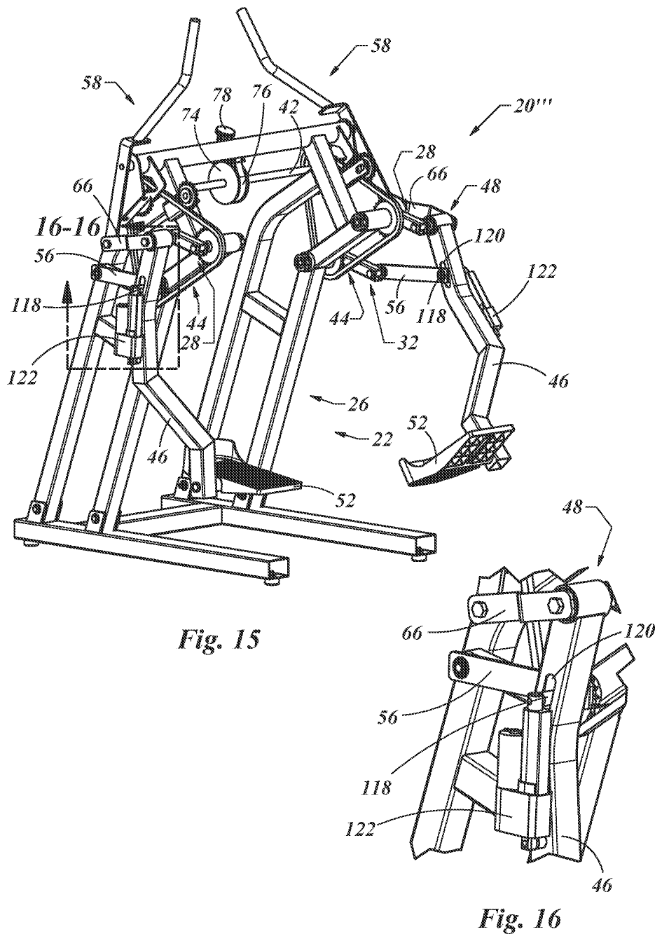

Another option to vary the stride length of the pedals 52 is presented in FIGS. 15-17. In this embodiment of the exercise device 20′″ the crank link 56 may be used, or the movable crank link 96 as disclosed previously. As previously noted, the movable crank link 96 may enable a system in which the user may alter the stride length by simply using a longer stride length in their gait and the exercise device 20″ may conform to the user's foot path of travel. In FIGS. 15-17 a system is illustrated which may provide a set stride length that is adjustable. It is possible to use both the movable crank link 96 as disclosed with the adjustable system illustrated in FIGS. 15-17. That combination is not shown though it is understood that this combination is inherently part of this disclosure.

With regard to FIGS. 15-17, an exercise device 20′″ is shown in which the crank link pin 54 of the previous embodiments may be replaced with a movable link pin 118. The movable link pin 118 may be slidably or pivotally mounted to the pedal arm 46. It is shown to be received by a slot 120 in the pedal arm 46, but this slot 120 is not critical. The movable link pin 118 may be mounted to the linear slide, or pivotally coupled to the pedal arm 46, in any way that is known in the art, so that a controlled movement of the movable link pin 118 may be provided. An actuator 122 may be used to move the movable link pin 118 on the pedal arm 46 closer to, or further from, the first end 48 of the pedal arm 46. If the movable link pin 118 is moved up closer to the first end 48 of the pedal arm 46, as illustrated by the up arrows 124, the stride length of the pedal 52 may be increased. If the movable link pin 118 is moved down, farther from the first end 48 of the pedal arm 46, as illustrated by the down arrows 126, the stride length of the pedal 52 may be decreased. This adjustment of the relative position of the movable link pin 118 as moved by the actuator 122 may be adjusted by the user before an exercise session begins or with a system that may be powered, such as with the actuator 122, the adjustment may be also accomplished while the user is performing the exercise. A simple screw adjustment or even a movable but locking movable link pin 118 system may be used where the user may adjust and lock the movable link pin 118 in a set position prior to beginning the exercise session.

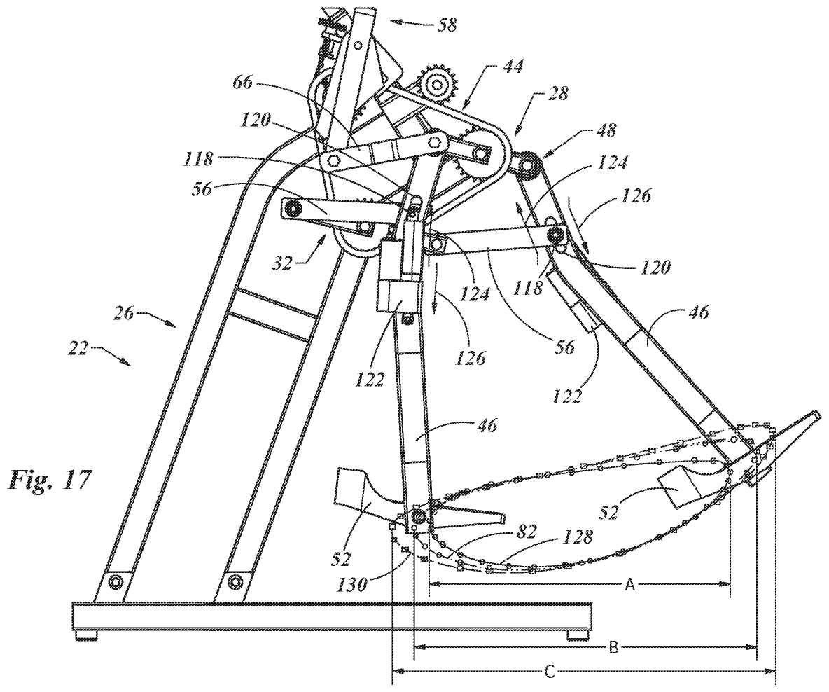

The horizontal displacement of multiple stride lengths of the pedals 52 that may be provided by varying the position of the movable link pin 118 is illustrated in FIG. 17. The trace of the pedal path 82, as previously presented, may be shown as a midpoint of the location of the movable link pin 118. This may provide a horizontal dimension of the trace of the pedal path 82 as identified by the value “B”. If the movable link pin 118 is moved in accordance with the down arrows 126, thereby increasing the distance between the movable link pin 118 and the first end 48 of the pedal arm 46, a shorter pedal path 128 may result. The shorter pedal path 128 may be associated with a smaller horizontal displacement identified as “A”. If the movable link pin 118 is moved closer to the first end 48 of the pedal arms 46, as noted by the up arrows 124, a greater stride length may result. The longer pedal path 130 may be associated with the greater horizontal displacement identified as “C”.

If the movable link pin 118 is used with a constant length crank link 56, as shown, the adjustment in the stride length may provide a series of set pedal paths (82, 128, 130) or any infinite number of variations to those shown. Each path may be a result of the settings of the exercise device 20′″ and therefore stable to the user, as the user may not be able to alter the pedal paths without making an adjustment to the position of the movable link pin 118. This stability may be desirable to some users in that their body may be fully supported on the pedals 52 of the exercise device 20′″. Using the movable crank link 96 with the adjustable stride length system as provided by the movable link pin 118 together may provide a system which allows for the user to vary their stride length where the path of the pedals 52 comply with that of the user, and an adjustable baseline path of the path of the pedals 52 may be provided by the movable link pin 118 and set in accordance with the desire or some physical characteristics of the user.

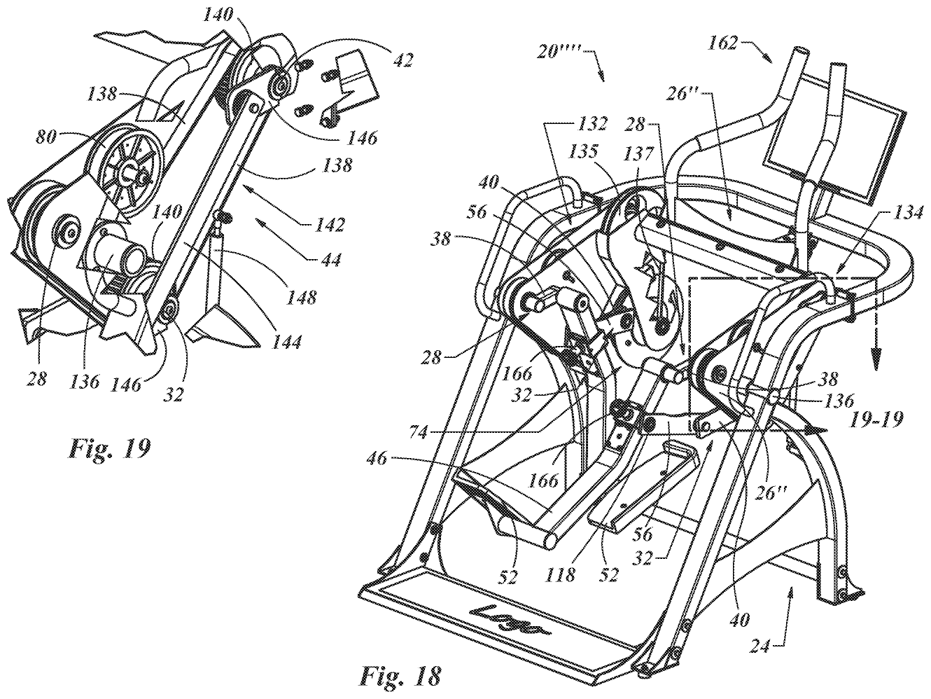

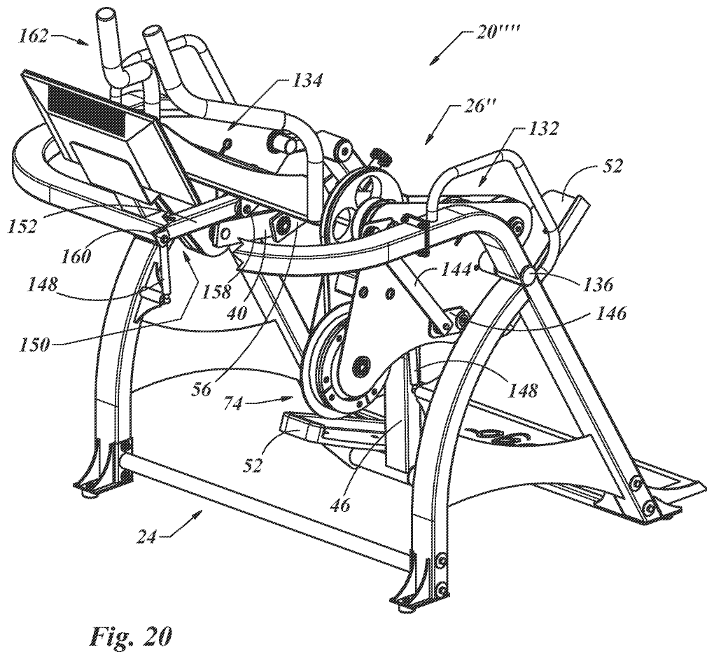

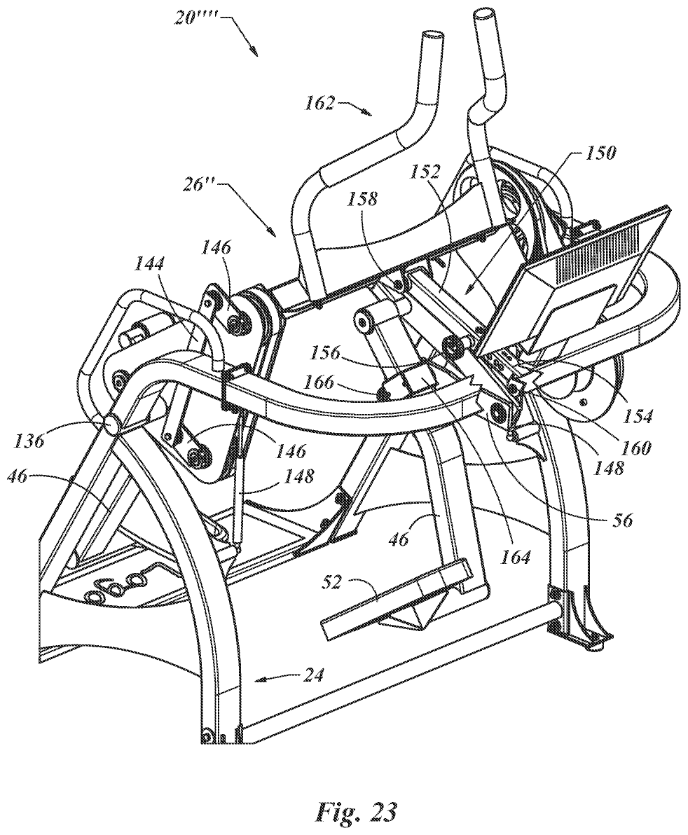

Another embodiment of the exercise device 20″″ is shown in FIGS. 18-23. With attention to FIGS. 18-20, the support frame 26″ may be comprised of first drive 132 and a second drive 134, each coupled to the support frame 26″. The support frame 26″ may be movably coupled to the base frame 24 by way of a pivot joint 136. The first drive 132 and the second drive 134 may be positioned such that a person may position themselves between the first drive 132 and the second drive 134. The support frame 26″ may also support the drive shaft 42, which may be in mechanical communication with the flywheel 74. The first drive 132 and the second drive 134 may each support a first crank assembly 28 and a second crank assembly 32 in such a manner such that the first crank arm 38 of the first crank assembly 28, coupled to the first drive 132 may be one hundred and eighty degrees out of phase from the first crank arm 38 of the first crank assembly 28, coupled to the second drive 134. In a similar manner, the second crank assembly 32 may include a second crank arm 40 coupled to the first drive 132 that may be one hundred and eighty degrees out of phase from the second crank arm 40, coupled to the second drive 134. The first crank arm 38 and the second crank arm 40 may be parallel to each other on both the first drive 132 and the second drive 134.

A drive pulley 135 may be mounted to the drive shaft 42. The drive pulley 135 may be in mechanical communication with the flywheel 74 by way of a drive belt 137. A braking system may be coupled to the flywheel 74 so that resistance provided to the flywheel 74 may offer a resistance to movement of the drive shaft 42, which in turn may offer a resistance to movement of the pedals 52. This combination may offer a form of exercise resistance to the user.

The control system 44 may include a drive member 138, which may take the form of a belt, roller chain, a synchronous belt, a v-belt, a poly-v belt or any other power transmission system known in the art. The drive member 138, as shown here in the form of a belt, may be tensioned by the idler 80. A drive pulley 140 may be secured to the first crank assembly 28 and the second crank assembly 32 on both the first drive 132 and the second drive 134. The drive member 138 may be limited to a belt or other power transmission system alone. Alternatively it may be advantageous to provide a secondary power transmission system in the form of a torque linkage 142. This may include a link rod 144 that may be coupled to the second crank assembly 32 and the drive shaft 42 by way of a pair of clamp links 146. This torque linkage 142 may be used to supplement the drive member 138 when high torque is applied to the second crank assembly 32 by the user. The braking system may be applied to the flywheel 74, as such, the highest load may be seen between the second crank assembly 32 and the drive shaft 42. The use of the torque linkage 142 may allow the drive member 138 to be designed for the lower torque associated with extended use by a user and when high forces are applied by the user, such as during sprinting or high incline “hiking” movements, the torque linkage 142 may supplement the power transmission, taking any excessive stress off the drive member 138. This may help eliminate the possibility of drive member 138 jumping a tooth of one of the drive pulleys 140 without the need to over engineer the drive member 138 and drive pulleys 140 for stresses that are only seen occasionally and for short durations.

The torque linkage 142 is shown here to connect the second crank assembly 32 to the drive shaft 42, which may be connected to the braking system. This combination may experience the highest forces and that is why it is shown in this configuration. It is understood that one or more torque linkages 142 may connect any one or more combinations between the first crank assembly 28, the second crank assembly 32 and the drive shaft 42.

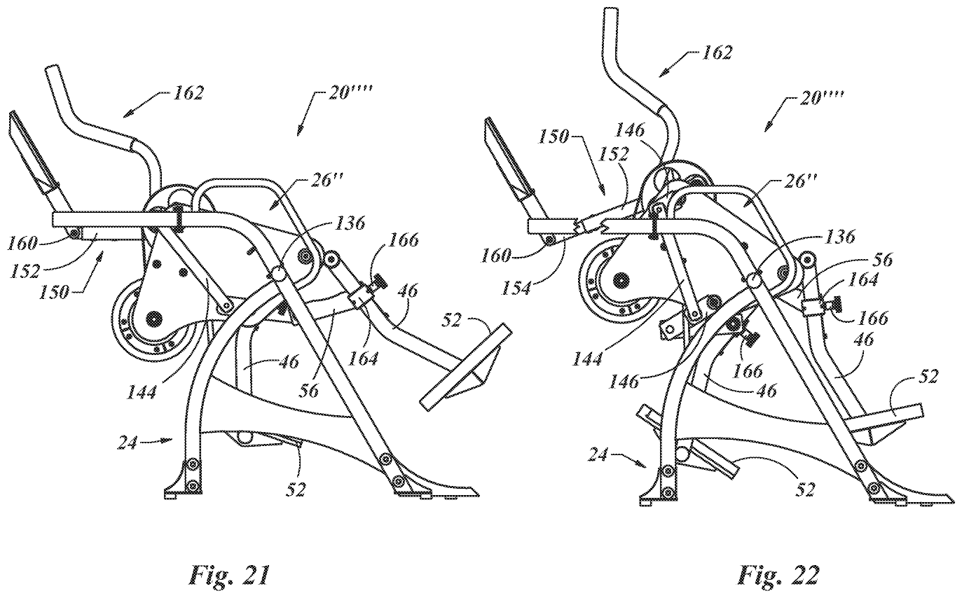

The transfer of the higher forces that may be added by the user to the torque linkage 142 may be generated by the user by positioning the exercise device 20″″ in a configuration so as to simulate walking up a hill. In this embodiment, this may be accomplished by altering the position of the support frame 26″ and all the elements supported by the support frame 26″ with respect to the base frame 24. One method of doing this is illustrated in FIGS. 21-23. The support frame 26″ may be pivotally coupled to the base frame 24 about the pivot joint 136. One or more counterbalance springs 148 may be coupled to the support frame 26″ and to the base frame 24 to counter any torque the support frame 26″ may provide at rest about the pivot joint 136 due to the weight of the support frame 26″ and the other elements coupled to the support frame 26″. This counterbalance spring 148 may be in the form of a gas spring. An actuator, such as a linear screw actuator, may be substituted for one or both of the counterbalance springs 148 shown here. In that embodiment, the actuator may drive the front portion of the support frame 26″ up or down and thereby change the angular orientation of the support frame 26″ relative to the base frame 24. By changing this orientation, the pedal path, as shown in previous Figures, may also change, as inclining the front of the support frame 26″ up may cause the pedal path to simulate walking up a hill.

To reduce production costs, compared to using linear actuators in place of the counterbalance springs 148, the gas springs may be used as the counterbalance springs 148 with an incline adjustment 150. The incline adjustment 150 may include a first support 152, which may articulate with a second support 154, and a locking pin 156 to releasably secure the first support 152 to the second support 154 at desired positions. This may securely alter the dimension between a support frame pin 158 and a base frame pin 160. By increasing the dimension between the support frame pin 158 and the base frame pin 160, exercise device 20″″ may produce an inclined pedal angle from flat (as shown in FIG. 21) to an inclined position (as shown in FIG. 22). The user may disengage the locking pin 156, move the support frame 26″ to a desired position, being aided by the counterbalance springs 148, and then engaging the locking pin 158 to secure the support frame 26″ and therefore the first drive 132 and the second drive 134, to a desired level of incline.

In this embodiment the drive handles 58 of previous embodiments have been removed and a set of stationary leaning handles 162 are shown. These handle types are not mutually exclusive to any embodiment. The leaning handles 162 may be desirable in some angular orientations of the exercise device 20 and the moving drive handles 58 may be desirable in other orientations, or as a personal preference in any orientation. Either form of handles (moving drive handles 58 or stationary leaning handles 162) may be interchangeably used or in combination together on any embodiment.

A method of altering the pedal path by varying the position of the crank link 56 on the pedal arm 46 has been disclosed. This embodiment of the exercise device 20″″ illustrates a manually adjustable version to accomplish this task. An adjustment bracket 164 may be releasably secured to the pedal arm 46 at one or more positions on the pedal arm 46. A leg lock pin 166 may be used to releasably secure the adjustment bracket 164 to a position on the pedal arm 46. The crank link 56 may be pivotally secured to the adjustment bracket 164 at the movable lock pin 118, and as noted before, also to the second crank arm 40. Therefore, by adjusting and securing the adjustment bracket 164 at different positions on the pedal arm 46, the path of the pedals 52 may be altered to achieve more than one pedal path, as previously disclosed.

The foregoing detailed description of the present invention is provided for purposes of illustration, and it is not intended to be exhaustive or to limit the invention to the particular embodiment shown. The embodiments may provide different capabilities and benefits, depending on the configuration used to implement key features of the invention.

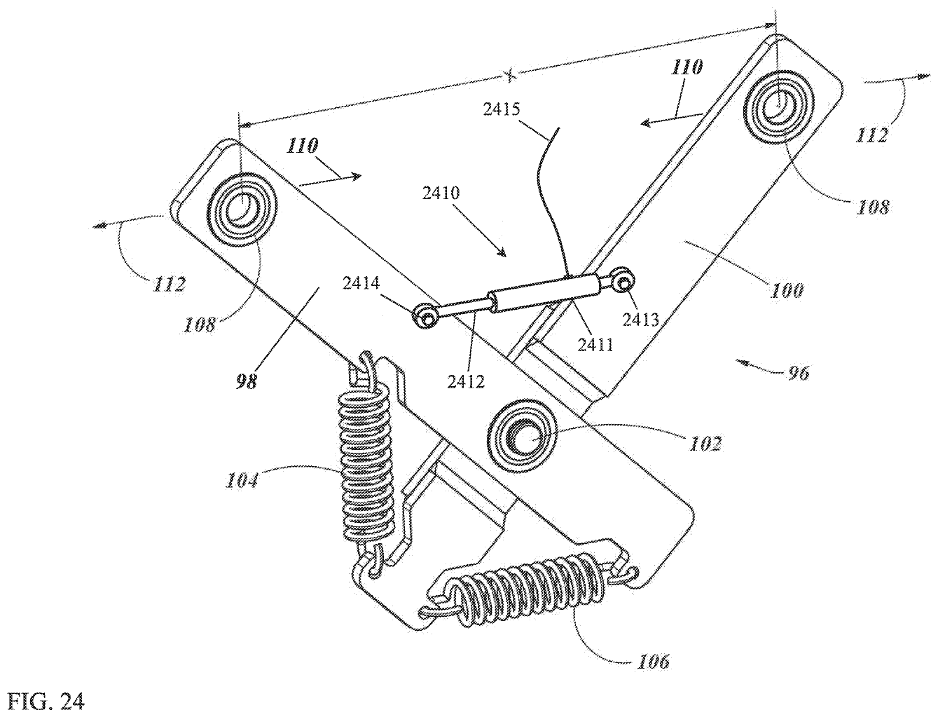

FIG. 24 is a diagram illustrating an exemplary sensor for detecting movement of a movable crank link. Movable crank link 96 shown in this diagram is the same as that shown and described for FIG. 12, but with the addition of a linear position sensor 2410 to provide data regarding the amount of movement of movable crank link 96 during operation. Linear position sensor may be of any suitable type including, but not limited to, analog or digital type, resistive type, inductive type, and variable differential transformer type. Linear position sensor of this embodiment comprises a body 2411 comprising a fixed operational sensor component (e.g., a resistor coil or an inductive coil) and a traveling arm 2412 comprising movable operational sensor component (e.g., an electrical contact for causing an electrical connection along the resistor coil or a magnet for causing an induction in the inductive coil). Body 2411 is affixed to one link 100 of movable crank link 96 via pivoting attachment 2413 and traveling arm 2412 is affixed to the other crank link 98 via a pivoting attachment 2414. Compression of the ends of movable crank link 96 in the direction shown at 110 or elongation of the ends of movable crank link 96 in the direction shown at 112 will cause traveling arm 2412 to push into, or pull out of, body 2411, causing a position signal to be transmitted via wires 2415 to an appropriate computing device for data capture, storage, and use. As the mechanical characteristics of exercise machine are known (e.g., lengths, attachment points, and attachment angles, of crank links 98, 100, crank arms 38, 40, pedal arms 46, etc.), changes in positional data from linear position sensor can be translated into useful exercise statistics such as stride length and stride period.

Many other configurations are possible including, but not limited to, mounting of the linear position sensor inside or alongside one or more of the springs 104, 106, or mounting of the linear position sensor at a position on the side of the crossed links opposite to spring 104. Other types of position sensors may be used in a manner similar to linear position sensors including, but not limited to, ultrasonic distance sensors, optical distance or position sensors, and strain gauges.

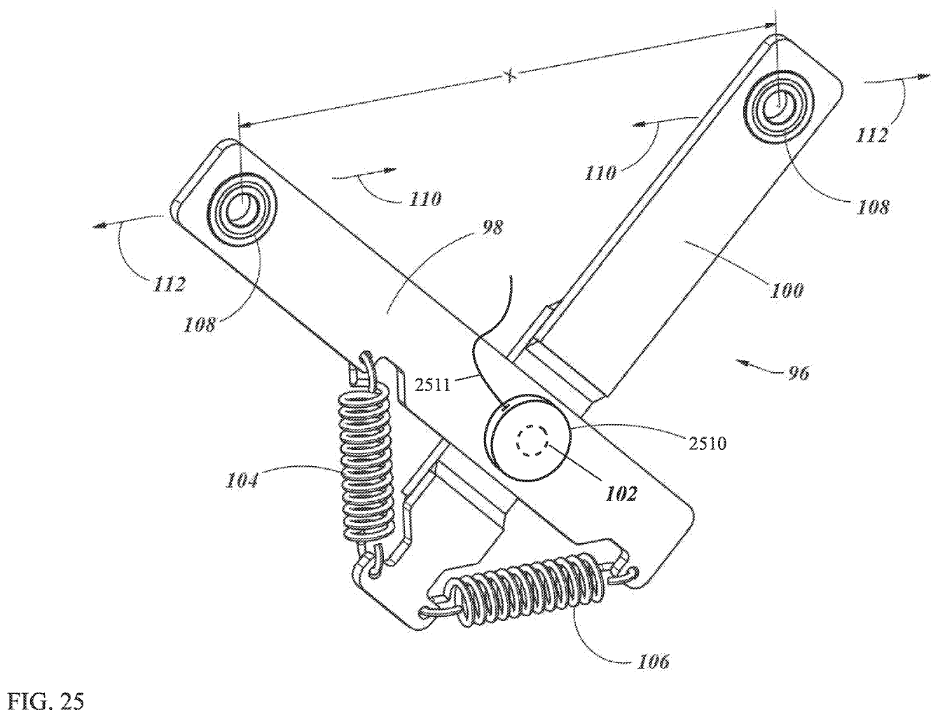

FIG. 25 is a diagram illustrating another exemplary sensor for detecting movement of a movable crank link. Movable crank link 96 shown in this diagram is the same as that shown and described for FIG. 12, but with the addition of an angular position sensor 2510 to provide data regarding the amount of movement of movable crank link 96 during operation. Angular position sensor may be of any suitable type including, but not limited to, analog or digital type, resistive type or optical type, and incremental position encoding type or absolute position encoding type. Angular position sensor of this embodiment comprises a body 2510 comprising a fixed operational sensor component (e.g., a resistor coil or an encoded optical disk) and a rotating shaft (not shown) affixed to pivot 102 of movable crank link 96. comprising movable operational sensor component (e.g., an electrical contact for causing an electrical connection along the resistor coil or a magnet for causing an induction in the inductive coil). Body 2510 is affixed to one link 98 of movable crank link 96 rotating shaft (not shown) is affixed to pivot 102 which, in this configuration must be affixed to the other crank link 100 such that movement of crank links 98, 100 causes rotating shaft (not shown) to rotate inside body 2510 proportionally to the movement of the crank links 98, 100. Compression of the ends of movable crank link 96 in the direction shown at 110 or elongation of the ends of movable crank link 96 in the direction shown at 112 will cause rotating shaft (not shown) to rotate within body 2510 either clockwise or counter-clockwise, causing a position signal to be transmitted via wires 2511 to an appropriate computing device for data capture, storage, and use. As the mechanical characteristics of exercise machine are known (e.g., lengths, attachment points, and attachment angles, of crank links 98, 100, crank arms 38, 40, pedal arms 46, etc.), changes in positional data from linear position sensor can be translated into useful exercise statistics such as stride length and stride period.

Many other configurations are possible including, but not limited to, mounting of the linear position sensor inside or alongside one or more of the springs 104, 106, or mounting of the linear position sensor at a position on the side of the crossed links opposite to spring 104. Other types of position sensors may be used in a manner similar to linear position sensors including, but not limited to, ultrasonic distance sensors, optical distance or position sensors, and strain gauges.

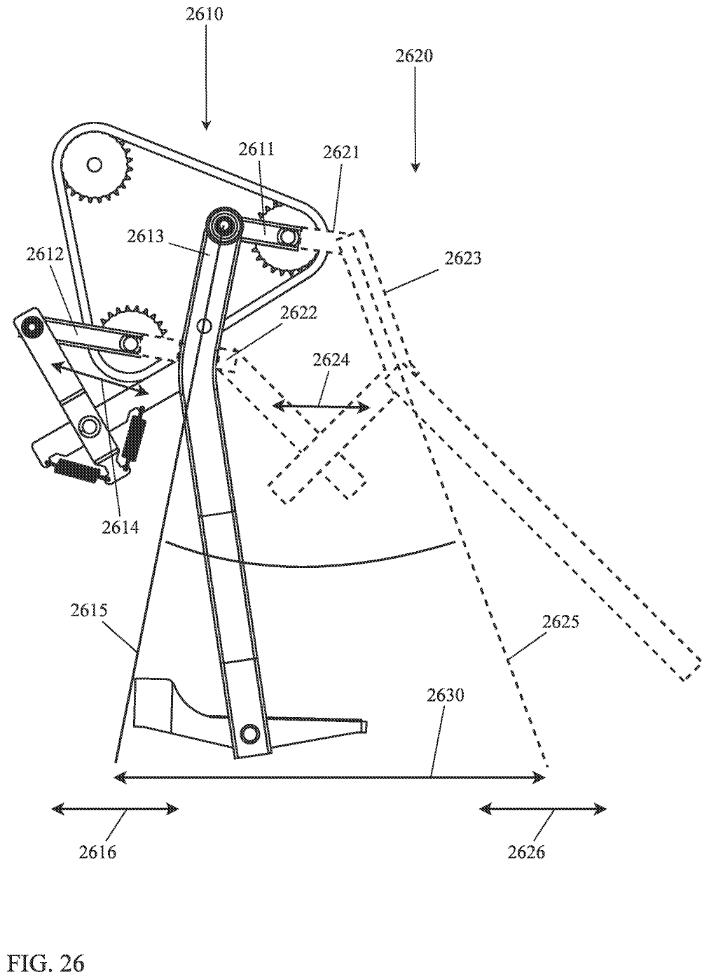

FIG. 26 is a diagram illustrating an exemplary method for calculation of stride length in an embodiment having movable crank link. As movement data is obtained from sensors place about exercise device 20, useful exercise statistics such as stride length and stride period can be calculated. As the mechanical characteristics of exercise machine are known (e.g., lengths, attachment points, and attachment angles, of crank links 98, 100, crank arms 38, 40, pedal arms 46, etc.), changes in positional data from position sensors located about exercise device 20 can be translated into useful exercise statistics such as stride length and stride period.

An example of such calculations is shown in this diagram. Here, a simplified view of a single side of the natural motion exercise mechanism is shown without frame 22. The components of the natural motion exercise mechanism are shown in a first position 2610 corresponding to rotation of crank arms 2611, 2612 in a forward position (shown in solid lines) and in a second position 2620 corresponding to rotation of crank arms 2621, 2611 to a backward position (180 degrees of rotation from the forward position; shown in dashed lines). As the length and angles of attachment of the various components of the natural motion exercise mechanism are known a stride length can be calculated as a base stride length 2630 from a forward-most position of the pedal 2615 in the first position 2610 to a backward-most position of the pedal 2625 in the second position, plus an offset at each end 2616, 2626 of base stride length corresponding to compression or extension of movable crank link 2614 in first position 2610 and compression or extension of movable crank link 2624 in second position 2620.

This principle of using sensor position information with known mechanical characteristics of exercise device 20 can be used to calculate other useful exercise statistics including, but not limited to, stride length variability, stride period variability, stride height, stride height variability, instantaneous or average power expended during exercise, and total work performed during exercise.

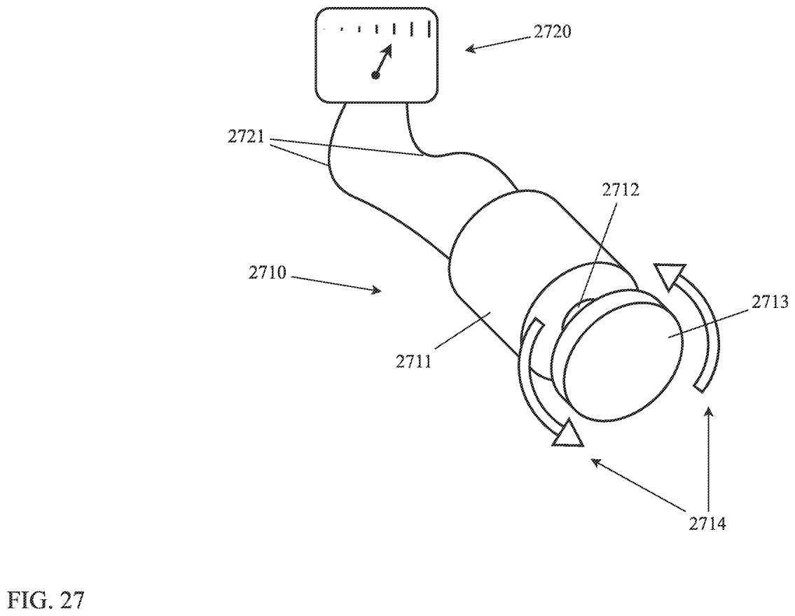

FIG. 27 is a diagram illustrating use of a direct current generator to determine power output of a user during exercise. Several means exist for capturing data related to power output of a user during exercise. A common method for determining power output on bicycles, for example, is to affix a strain gauge to the crank arms of the cranks (or integrate the strain gauge in to the crank arms), take a reading of the force applied to the crank arms during use, and calculate the power applied to the pedals during each rotation of the cranks using the force readings. The same method could be applied to exercise device 20 by affixing a strain gauge to some component of the natural motion exercise mechanism, such as the pedal arms, and making similar calculations. The method shown here uses a direct current (DC) generator 2710 comprising a generator housing having internal induction coils as in a typical DC motor, a drive shaft 2712 having permanent magnets arranged to generate an electrical current in the induction coils during rotation of the drive shaft, and a pulley or gear 2713 engaged with the belt or chain of control system 44. Rotation of control system 44 causes the belt or chain (e.g., belt 137) engaged with pulley or gear 2713 to rotate pulley or gear 2713, generating a current along wires 2721, measurable by a current meter 2720. Using the known electrical characteristics of DC generator such as the current known to be generated by DC motor as various revolution per minute (RPM) speeds, combined with the known characteristics of exercise device 20 such as the length of the pedal arms and the known resistance to movement of its braking system at its current braking setting, the measured current can be converted into an instantaneous power output in watts. In other embodiments, particularly those in which the braking system comprises a electromagnetic resistance mechanism, the power might be measured directly from the braking system as a proportion of the current flowing through the electromagnetic resistance mechanism (which is generally proportional to the rotation speed of the shaft or disc of the electromagnetic resistance mechanism).

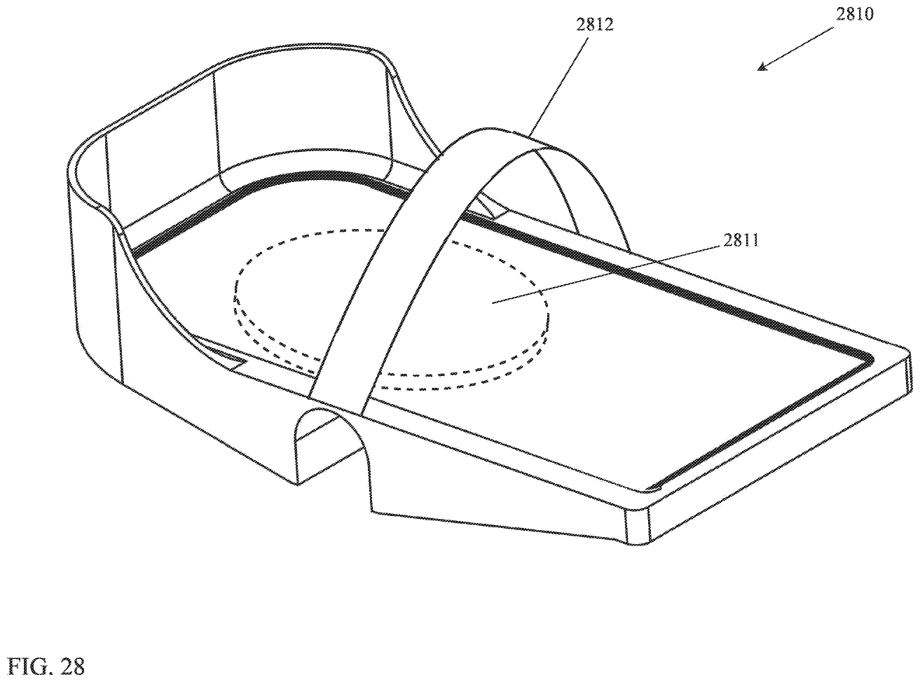

FIG. 28 is a diagram illustrating an exemplary footpad for detecting pressure and lift of a user's foot during exercise. For some applications, such as walking or running gait detection or analysis, it is useful to have sensor data showing a downward pressure applied by a user's foot onto a footpad or pedal or an upward lift pressure of the user's foot away from the footpad or pedal. Here, an pressure sensor 2811 is shown integrated into a footpad or pedal 2810, along with a strap 2812 configured to rest across the top of a user's foot.

Pressure sensor 2811 is configured to measure a downward pressure applied by a user's foot onto footpad or pedal 2810 which can be sent to a computing device as pressure data comprising a magnitude of the force applied, the speed at which the force is applied, a time of application of the force, etc., which pressure data can be captured over time to provide additional information such as a period of application of force. Depending on configuration, pressure sensor 2811 may be of various configurations such as having a resistive, capacitive, piezo-electric, or optical, or other force sensor technology.

Strap 2812 is configured to measure an upward lift pressure of the user's foot away from the footpad or pedal applied by the top of a user's foot against strap 2812 which can be sent to a computing device as pressure data comprising a magnitude of the force applied, the speed at which the force is applied, a time of application of the force, etc., which pressure data can be captured over time to provide additional information such as a period of application of force. Depending on configuration, strap 2812 may be of various configurations such as having a strain gauge integrated into its material, having a pressure sensor affixed to the underside of strap 2812, or having one or both ends of strap affixed to a strain gauge or force sensor.

In another configuration (not shown), footpad or pedal 2810 may be configured on a rotational platform with a rotary encoder or other mechanism for determining the angle of rotation of the user's foot during exercise.

Data from the above-described pressure sensors and rotational sensors can be used to calculate useful exercise statistics including, but not limited to, a periodicity of a user's gait, variations in the periodicity of a user's gait, discrepancies in sided-ness of the user's gait (e.g., indicating a preference for right side over left side or vice-versa, possibly indicating a disability or movement restriction), changes in, or discrepancies in, a user's foot angle or rotation during certain exercises or motions, or discrepancies in sided-ness of the user's foot angle or rotation (again, possibly indicating some disability or movement restriction).

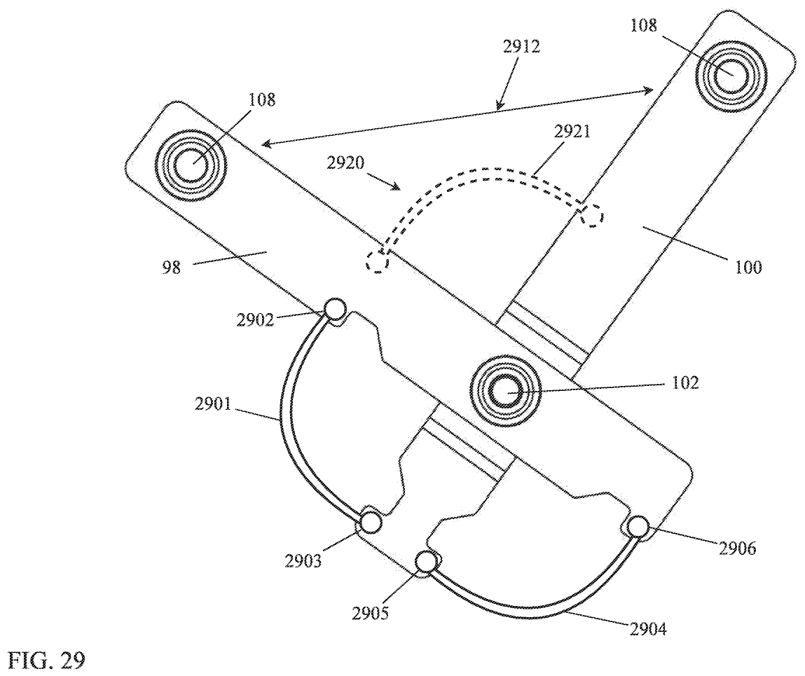

FIG. 29 is a side elevation view of a movable crank link utilizing leaf springs for variable stride length resistance. As described above in relation to FIG. 12, movable crank link 96 allows the stride length of exercise device to naturally adjust to a user's stride length by virtue of compression of attachment points 108 toward one another at one end of the stride and/or expansion of attachment points 108 away from one another at the other end of the stride, allowing the pedals to move further forward or backward than they would with a fixed distance 2912 between attachment points 108. This compression and/or expansion occurs naturally as the user drives pedals forward and backward exceeding toward each end of the stride the neutral position of movable crank link 96 (i.e., without force applied by the user during a stride).

Movable crank link 96 as shown and described is not limited to coiled springs. Any suitable force resistance device or mechanism may be used including, but not limited to, coiled or wound springs, flat or leaf springs, torsion bars, elastomeric materials such as rubber and synthetic polymers capable of deformation and return to their original form after deformation. The chosen resistance mechanism or mechanisms may be configured to resist pulling, pushing, or twisting, and its location on and attachment to movable crank link 96 may differ depending on that configuration, so long as they allow for compression, elongation, or both, of a distance 2912 between two attachment points 2910, 2911 of movable crank link 96 when force is applied and a tendency to return to the original distance when the force is removed.

Here, leaf springs 2901, 2904 are substituted for coiled springs of FIG. 12. Leaf springs, also known as flat springs, laminated springs, and carriage springs, depending on their application, are lengths of a flexible material which bend to provide resistance. They are typically flat, thin bars of material formed into an arc shape. They are typically made of spring steel, but any suitable material may be used including, but not limited to, other metals, plastics, and even wood. Leaf springs of this embodiment are shaped with a strong arc and attached at their ends 2902, 2903, 2905, 2906 between first link 98 and second link 100, providing resistance to both compression of, and expansion of, the distance 2912 between attachment points 108, and a tendency to return to their original shape when force is removed. The strong arc allows not only for compression of each leaf spring 2901, 2902 between their respective attachment points, but also expansion between their respective attachment points.

An example of an alternate location of, and configuration of, a leaf spring of this example is shown at 2920. Wherein a single leaf spring 2921 is attached between first link 98 and second link 100 at a point on each link between the attachment points 108 and the pivot point created by crank link pin 102.

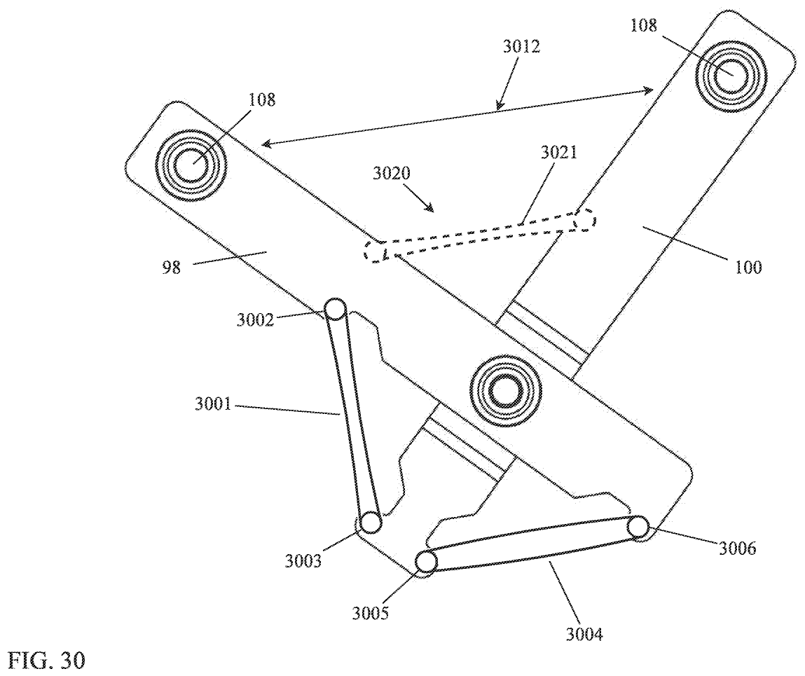

FIG. 30 is a side elevation view of a movable crank link utilizing elastomeric bands for variable stride length resistance. As described above in relation to FIG. 12, movable crank link 96 allows the stride length of exercise device to naturally adjust to a user's stride length by virtue of compression of attachment points 108 toward one another at one end of the stride and/or expansion of attachment points 108 away from one another at the other end of the stride, allowing the pedals to move further forward or backward than they would with a fixed distance 3012 between attachment points 108. This compression and/or expansion occurs naturally as the user drives pedals forward and backward exceeding toward each end of the stride the neutral position of movable crank link 96 (i.e., without force applied by the user during a stride).

Movable crank link 96 as shown and described is not limited to coiled springs. Any suitable force resistance device or may be used including, but not limited to, coiled or wound springs, flat or leaf springs, torsion bars, elastomeric materials such as rubber and synthetic polymers capable of deformation and return to their original form after deformation. The chosen resistance mechanism or mechanisms may be configured to resist pulling, pushing, or twisting, and its location on and attachment to movable crank link 96 may differ depending on that configuration, so long as they allow for compression, elongation, or both, of a distance 3012 between two attachment points 3010, 3011 of movable crank link 96 when force is applied and a tendency to return to the original distance when the force is removed.

Here, elastomeric bands 3001, 3004 are substituted for coiled springs of FIG. 12. elastomeric bands are bands, tubes, rods, or other lengths of a flexible material which deform by elongation or compression to provide resistance. They are typically made of rubber or synthetic polymers. Elastomeric bands 3001, 3004 of this embodiment are cylindrical rods of synthetic polymer, attached at their ends 3002, 3003, 3005, 3006 between first link 98 and second link 100, providing resistance to both compression of, and expansion of, the distance 3012 between attachment points 108, and a tendency to return to their original shape when force is removed. Elastomeric bands 3001, 3004 stretch under pulling force as shown at 3001 (showing elastomeric band 3001 stretched and thinner in the middle section) and compress (or bend) under compressive force as shown at 3004 (showing elastomeric band 3004 bulging slightly in the middle section; further compression would cause elastomeric band 3004 to bend).

An example of an alternate location for elastomeric band 3001 is shown at 3020, wherein elastomeric band 3001 is attached between first link 98 and second link 100 at a point on each link between the attachment points 108 and the pivot point created by crank link pin 102.

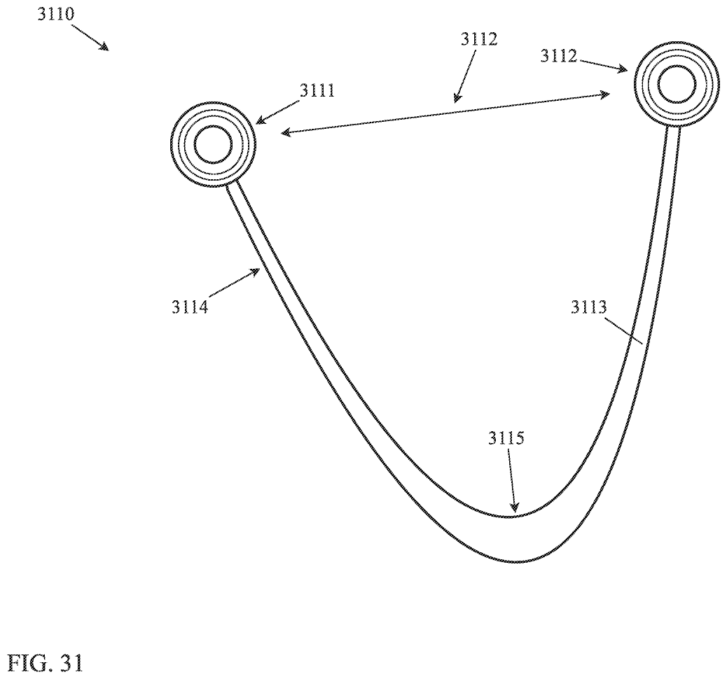

FIG. 31 is a side elevation view of a single-piece movable crank link utilizing a single leaf spring for variable stride length resistance. As described above in relation to FIG. 12, movable crank link 3110 allows the stride length of exercise device to naturally adjust to a user's stride length by virtue of compression of attachment points 3111, 3112 toward one another at one end of the stride and/or expansion of attachment points 3111, 3112 away from one another at the other end of the stride, allowing the pedals to move further forward or backward than they would with a fixed distance 3112 between attachment points 3111, 3112. This compression and/or expansion occurs naturally as the user drives pedals forward and backward exceeding toward each end of the stride the neutral position of movable crank link 3110 (i.e., without force applied by the user during a stride).

Movable crank link 3110 as shown and described is not limited to a leaf spring. Any suitable force resistance device or may be used including, but not limited to, coiled or wound springs, flat or leaf springs, torsion bars, elastomeric materials such as rubber and synthetic polymers capable of deformation and return to their original form after deformation. The chosen resistance mechanism or mechanisms may be configured to resist pulling, pushing, or twisting, and its location on and attachment to movable crank link 3110 may differ depending on that configuration, so long as they allow for compression, elongation, or both, of a distance 3112 between two attachment points 3110, 3111 of movable crank link 3110 when force is applied and a tendency to return to the original distance when the force is removed.

Here, a single leaf spring 3113 is substituted for the mechanism of FIG. 12. Leaf springs, also known as flat springs, laminated springs, and carriage springs, depending on their application, are lengths of a flexible material which bend to provide resistance. They are typically flat, thin bars of material formed into an arc shape. They are typically made of spring steel, but any suitable material may be used including, but not limited to, other metals, plastics, and even wood. Leaf spring 3113 of this embodiment is shaped with a strong arc and attached at its ends to attachment points 3111, 3112 providing resistance to both compression of, and expansion of, the distance 3116 between attachment points 3111, 3112, and a tendency to return to its original shape when force is removed. Leaf spring 3113 of this embodiment has a thicker section in its middle 3115 to provide resistance from breakage as the curved portion is subjected to the most stress, and with thinner sections at its ends 3114 to provide additional flexibility. The materials and dimensions of leaf spring 3113 can be changed to increase or decrease resistance. The strong arc allows not only for compression leaf spring 3113 between its respective attachment points 3111, 3112, but also expansion between those attachment points.

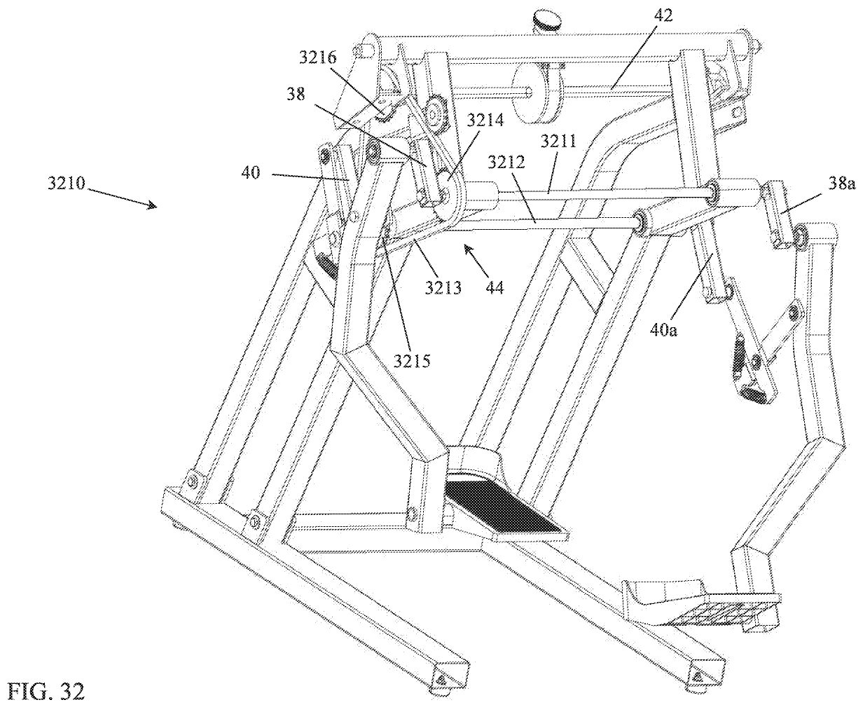

FIG. 32 is a diagram illustrating an embodiment of an exercise device having a single synchronized control system. Exercise device 3210 of this embodiment has similar characteristics as those described in other embodiments above. However, in this embodiment, only a single control system 44 is used, in this case having the same characteristics for control system 44 described above in other embodiments. However, in this case, instead of transferring the synchronized motion to a second control system 44 on the opposite side of exercise device 3210 via drive shaft 42, transfer of synchronized motion to the opposite side of exercise device 3210 is performed by a plurality of drive shafts 3211, 3212, each being connected in a fixed orientation with a crank arm 38, 40 and corresponding sprocket or pulley 3214, 3215 to the opposite of exercise device 3210. Crank arm 38 and sprocket/pulley 3214 are connected in a fixed orientation with drive shaft 3211 which is connected in a fixed orientation with crank arm 38a on the opposite side, wherein crank arm 38 and crank arm 38a are aligned 180 degrees opposite one another about the longitudinal axis of drive shaft 3211. Crank arm 40 and sprocket/pulley 3215 are connected in a fixed orientation with drive shaft 3212 which is connected in a fixed orientation with crank arm 40a on the opposite side, wherein crank arm 40 and crank arm 40a are aligned 180 degrees opposite one another about the longitudinal axis of drive shaft 3212. As in other embodiments, a drive belt/chain 3213 provides synchronized motion to a sprocket/pulley 3216 which is in a fixed orientation with drive shaft 42. While control system 44 of this embodiment is shown as being on one side of exercise device 3210, control system 44 may be located on the other side or in any suitable location, such as in the center of exercise device 3210.

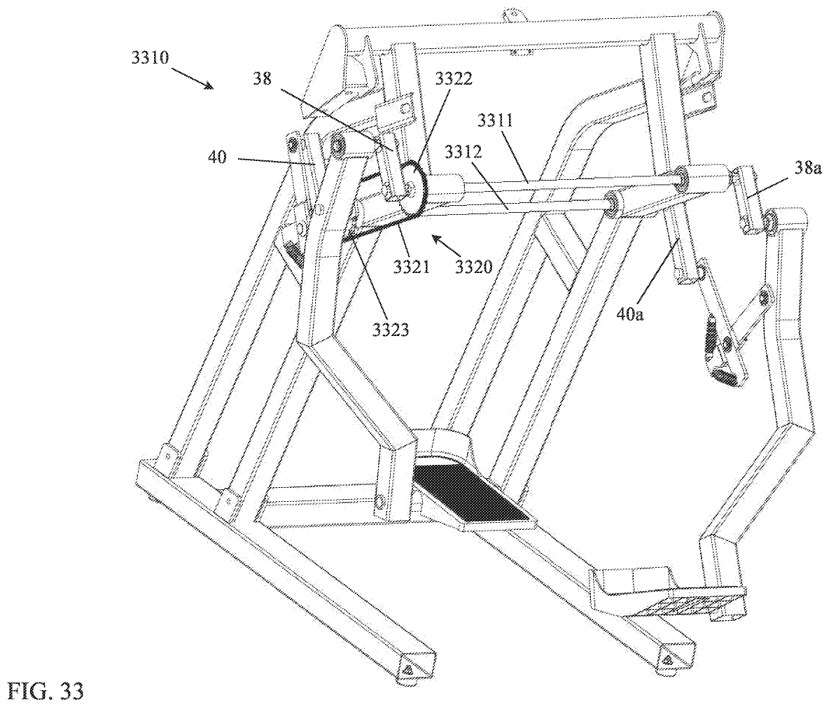

FIG. 33 is a diagram illustrating another embodiment of an exercise device having a single synchronized control system. Exercise device 3310 of this embodiment has similar characteristics as those described in other embodiments above. However, in this embodiment, only a single control system 3320 is used, in this example synchronizing only movement of pedal arms (and not an auxiliary system like drive shaft 42 and its resistance mechanism). In this case, instead of transferring the synchronized motion to a second control system on the opposite side of exercise device 3310 via drive shaft 42, transfer of synchronized motion to the opposite side of exercise device 3210 is performed by a plurality of drive shafts 3311, 3312, each being connected in a fixed orientation with a crank arm 38, 40 and corresponding sprocket or pulley 3322, 3323 to the opposite of exercise device 3310. Crank arm 38 and sprocket/pulley 3322 are connected in a fixed orientation with drive shaft 3311 which is connected in a fixed orientation with crank arm 38a on the opposite side, wherein crank arm 38 and crank arm 38a are aligned 180 degrees opposite one another about the longitudinal axis of drive shaft 3311. Crank arm 40 and sprocket/pulley 3323 are connected in a fixed orientation with drive shaft 3312 which is connected in a fixed orientation with crank arm 40a on the opposite side, wherein crank arm 40 and crank arm 40a are aligned 180 degrees opposite one another about the longitudinal axis of drive shaft 3312. As in other embodiments, a drive belt/chain 3321 provides synchronized motion between sprocket/pulley 3322 and sprocket/pulley 3323. While control system 3320 of this embodiment is shown as being on one side of exercise device 3310, control system 3320 may be located on the other side or in any suitable location, such as in the center of exercise device 3310.

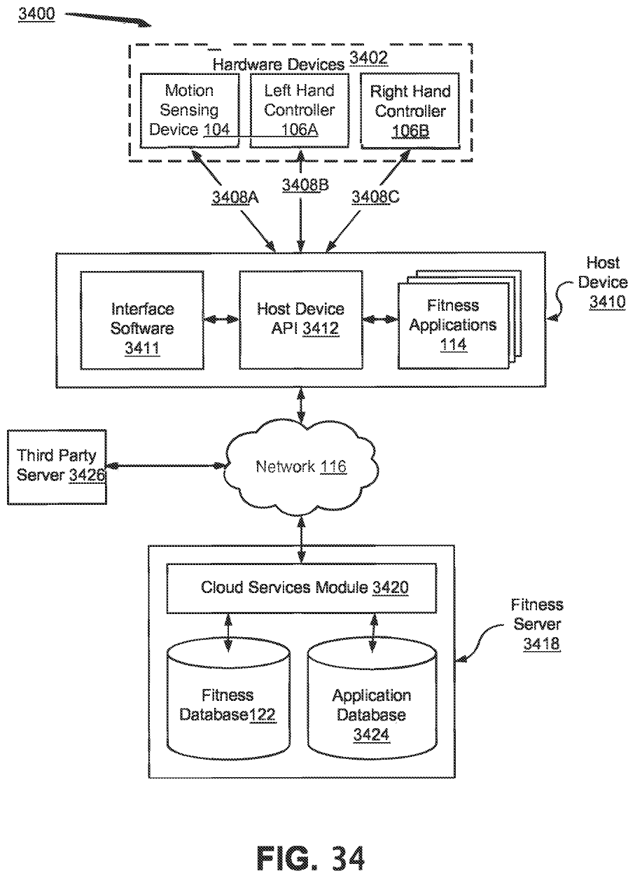

FIG. 34 is a block diagram illustrating components of the adaptable fitness system 100, according to one embodiment. The adaptable fitness system 100 includes a set of hardware devices 102, a host device 110, and a fitness server 118. In one embodiment, the hardware devices 102 generate a plurality of communication links 108A, 108B and 108C based on a user's interaction with the hardware devices 102. The host device 110 receives a plurality of data over the communication links 108A, 108B and 108C from the hardware devices 102 and interacts with a fitness application 114 or a plurality of fitness applications 114 based on the host devices' 110 interpretation of the data. The host device 110 communicates with the fitness server 118 over a network 116. In one embodiment, the hardware devices 102 include a motion sensing device 104, a left hand controller 106A and a right hand controller 106B. The motion sensing device 104 captures a plurality of motion data in real-time, as the user interacts with the fitness system 100. The left hand controller 106A receives input from the user by way of an action, such as the user pressing a button on the controller or moving a joystick on the controller. The right hand controller 106B receives input from the user by way of an action, such as the user pressing a button on the controller or moving a joystick on the controller. In alternative embodiments, additional hardware devices 102 may be used. For example, the hardware devices 102 may also include athletic shoes with embedded sensors or motion sensors that attach directly to the fitness system. In addition, some of the hardware devices shown 102 shown in FIG. 34 may be omitted. For example, only one of the hand controllers may be used, or the two hand controllers 106A, 106B may be replaced with a combined hand controller that contains the all of the buttons that would normally be distributed between the two hand controllers 106A, 106B. In some embodiments, multiple sets of hardware devices 102 may be added to the system 100 to adapt additional fitness devices into additional game controllers. For example, multiple stationary bicycles may be adapted into controllers for a bicycle racing application, thus allowing multiple users to participate in virtual races against each other.

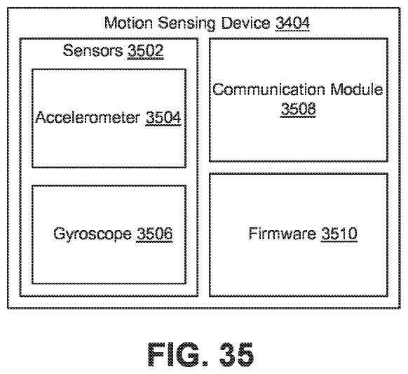

As a whole, the hardware devices 102 can be used to adapt a fitness device (e.g., a step machine, an elliptical machine, a treadmill, a rowing machine, etc.) into a game controller that receives a user's physical input (both motions and button presses) and generates corresponding input data for interacting with a fitness application 114. For example, the input data could be used to control a virtual character or object in the fitness application 114. Input data from the hardware devices 102 are sent over communication links 108A, 108B, 108C (hereinafter referred to collectively as 108) to the host device API 112 on the host device 110. The communication links 108 may be wired or wireless connections. In one embodiment, the communications links 108 are based on the Bluetooth Low Energy (BLE) protocol, and the hardware devices 102 are BLE devices. If additional sets of hardware devices 102 are included in the system, additional communication links 108 are established to connect the hardware devices 102 to the host device 110. In the illustrated embodiment, the communication links 108 connect each hardware device 102 directly to the host device 110. In this embodiment, the three hardware devices 102 connect simultaneously to the host device 110 over the communication links 108A, 108B, 108C. The hardware devices 102 may all connect over the same communication protocol (e.g., Bluetooth Low Energy), or over different communication protocols. In another embodiment, only the motion sensing device 104 is connected directly to the host device 110 via the first communication link 108A. In this embodiment, the two hand controllers 106A, 106B are coupled to the motion sensing device 104 via the other two communication links 108B, 108C, and the motion sensing device 104 is configured to relay input data from the hand controllers 106A, 106B to the host device 110. This embodiment is described in more detail with reference to FIG. 35. The host device 110 includes interface software 111, a host device API 112, and one or more fitness applications 114. As a whole, the host device 110 is a computing device that is capable of executing program instructions. The host device 110 may be, for example, a smartphone, tablet computer, laptop computer, or desktop computer. The interface software 111 and the host device API 112 operates in conjunction with the interface software to act as an interface between the hardware devices 102, the fitness applications 114, and the fitness server 118. When no hardware devices 102 are connected, the host device API 112 initiates a scan for hardware devices 102 at fixed intervals. Button presses on the hand controllers 106A, 106B or motion on the motion sensing device 104 will trigger the respective device to broadcast for connection (if not already connected). If the device's broadcast overlaps with one of the host device API's scanning intervals, then the host device API 112 initiates a connection attempt to the respective device. This process of establishing a connection after detecting a button press or motion allows for intuitive connection of hardware devices 102 to the host device 110. The host device API 112 maintains the connection between a fitness application 114 and a hardware device 102 as long as the application 114 remains active. After establishing a connection with one or more hardware devices 102, the interface software 111 and host device API 112 receive and process input data from the hardware devices 102 so that the input data can be used to control an application 114 or an object in an application 114. For example, the input data can be used by the application 114 to control a virtual character or object in a game. The host device API 112 can distinguish the input data from the different hardware devices because the firmware of each hardware device can be configured to include an identifier (e.g., a binary value) in a header of any input data that is sent to the host device 110. Thus, the motion sensing device 106 would have a different identifier than the hand controllers 106A, 106B. The interface software 111 can then build a software state representation of each device based on the device's identifier, and the host device API 112 can map input data to its respective software state representation to identify the originating hardware device. After the input data is mapped, e.g., via a table or index, to the corresponding hardware device, the data can be used to as inputs to fitness applications 114. In embodiments where the motion sensing device 104 is configured to relay input data from the hand controllers 106A, 106B to the host device 110, the motion sensing device 104 may be configured to read the identifier in the input streams from the two hand controllers 106A, 106B and relay a single integrated input stream to the host device 110. This process is described in more detail with reference to FIGS. 2A and 2D. In some embodiments, the left and right hand controllers 106A, 106B are manufactured to be identical (e.g., to reduce manufacturing costs) and have the same identifier. In these embodiments, it may be difficult to automatically distinguish between the input data from the two hand controllers 106A, 106B, so the interface software 111 may prompt the user to manually identify the two hand controllers (e.g., by first pressing a button on the left hand controller 106A and then pressing a button on the right hand controller 106B.) This setting can then be stored by the interface software 111 as part of the device's software state representation. The interface software 111 can use a similar process to distinguish input data from different hardware devices when multiple sets of hardware devices are connected to the host device 110 (e.g., for a multiplayer game). For example, the interface software 111 may display a prompt to move a first motion sensing device 104 (e.g., attached to a stationary bicycle), then display a prompt to move a second motion sensing device 104 (e.g., attached to an elliptical trainer), and then display similar prompts to identify the respective hand controllers 106 on the two fitness devices. In one embodiment, signals from the hand controllers 106A, 106B and motion sensing device 104 are wirelessly sent to the host device 110 as input data using a custom Bluetooth GATT profile. The data sent from the hardware devices 102 are then processed by the host device API 112. An example of the dataflow between the hardware devices 102 and the host device 110 is presented below: 1. The motion sensing device 104 acts as a server and the host device 110 connects as a client. 2. The host device 110 (the client) subscribes to data notifications of a specific service feature of the motion sensing device 104, such as an accelerometer. 3. A reading from the service feature (e.g., an accelerometer reading) is taken from motion sensing device 104. 4. The reading is sent to the host device 110 (the client subscriber). For example, the three-axis accelerometer reading may be sent as three bytes. 5. The interface software 111 decodes the reading and makes the readings available to applications 114 via the API 112. The readings are interpreted as input data by the application 114 and allow users to make selections or to control/manipulate elements (e.g., objects, characters, etc.) within the application 114 or to react to game cues or other interactive elements within the application 114. The fitness applications 114 are applications that receive input data from the hardware devices 102 through the host device API 112 and carry out some sort of user interaction based on the input data. For example, the fitness applications 114 can be exercise games or any non-game application that can interact with a fitness device in a meaningful way (e.g., an application 114 interact with hardware devices 102 mounted on a treadmill to simulate a walk through a park). The applications 114 contain one or more objects. In one embodiment, the object is a graphical representation such as a car, a ball or a character. The object also exhibits a controlled behavior, such as the graphical representation of the ball may exhibit a behavior of rolling. As the user performs the fitness activity the graphical representation or the object exhibits a controlled behavior based on the fitness activity performed by the user. For example, in one application the object can be a character representing the user. The character may exhibit a controlled behavior of running across the screen. The controlled behavior of running across the screen may be related to the speed at which the user runs. The faster the user runs the faster the character runs across the screen. Hence, the system allows the user to change and control the behavior of the object based on, in one instance, the intensity at which a user performs the fitness activity. In another example the object is a set of numbers on a scoreboard. The number may represent a number of metrics, such as distance run by the user or the number of calories burned by the user. The numbers on the scoreboard exhibit a controlled behavior of changing at a rate related to the speed at which the user is running at. Hence the faster the user runs the rate at which the numbers on the scoreboard change increases. In one embodiment, the input data received by the application 114 from the hardware devices 102 affects the behavior applied to the object in an application. Returning to the example of a character representing the user running across the screen; the user may press the button on the hand controller 106 causing the jumping behavior to be applied to the user's character in the application 114. This results in the user's character jumping over an obstacle as the user is playing the game. Hence, the user may also use the hardware devices 102 to apply a behavior to an object of the application 114. Applications 114 (including games) may be configured to encourage the user to exercise. As the user exercises, data received from the hardware devices 102 is recorded on the host device 110 to reflect progress in the user's experience. Applications 114 can also connect to a fitness server 118 through the network 116, where user data and statistics can be stored and accessed. One example of how the hardware devices 102 can interact with the software on the host device 110 could be a user attaching the motion sensing device 104 to the pedal of a stationary bicycle. An accelerometer in the motion sensing device 104 measures the motion of the pedals, allowing for realistic control of a ‘virtual biking’ application 114. Another example of how hardware devices 102 interact with software could be a user attaching the motion sensing device 104 to their hip, with the motion sensing device 104 detecting that motion for a dance or exercise game. The host device API 112 may be configured to run on any suitable operating system, such as iOS, Windows/Mac/Linux or Android. Different versions of the host device API 112 may be created for different operating systems. Although shown as a separate entity within the host device 110, the host device API 112 is typically a separate code library that is compiled into a fitness application 114 at build time. This allows a third-party developer can write a fitness application 114 that uses functions in the host device API 112 to exchange data with the hardware devices 102 and the fitness server 118. In one embodiment, the fitness server 118 is comprised of a cloud services module 120, a fitness database 122 and an application database 124. The fitness server 118 and its components 120, 122, 124 may be embodied as a single device or as a cluster of networked devices (e.g., a cluster of web servers). In one embodiment, the cloud services module 120 provides an interface over the network 116, by which the host device 110 can access fitness data from the fitness database 122 and application data from the application database on the fitness server 118. The cloud services module 120 receives historical data, such as physical activity and game performance data, from the host device API 112 and stores the data in the appropriate database 122, 124. For example, on installation of the fitness application 114 on a host device 110 running an Android operating system, the fitness application 114 may have to retrieve Android specific configuration and system commands from the application database 124 on the fitness server 118. In another example, the interface software 111 stores fitness data with respect to a specific user in the fitness database 122 on the fitness server 118. The user can now access the fitness data 122 on multiple host devices 110. In one embodiment, the cloud services module 120 also allows users to access their information on any device and at any time. The module also processes user requests to compare and share scores on third-party servers 126, such as social networking systems, e.g., FACEBOOK and TWITTER. The databases 122, 124 may be stored across a cluster of several servers for scalability and fault-tolerance. The cloud services module 120 can also host multiplayer games. In one embodiment, games are hosted in asynchronous context. After a user set ups a challenge, the cloud services module 120 can give a user a window in time to finish his “turn” or just wait until he completes a given goal. This information is stored in the application database 124. The cloud services module 120 executes the logic of the games, challenges, or goals that are created between players and maintains their states and progression. In an alternative embodiment, the cloud services module 120 also hosts multiplayer games in real-time, with real-time game data moving back and forth between players on different host devices 110. In another embodiment, a third party server 126 may communicate with the host device 110 or the fitness server 118 over the network 116. For example, the cloud services module 120 retrieves a user's social graph from a social networking system on a third-party server 126 and reproduces the social graph on the fitness server 118. Thus, subsequent functions associated with social game mechanics (e.g., the creation of challenges and cooperative goals) can be executed on the fitness server 118.

FIG. 35 is a block diagram illustrating the components of the motion sensing device 104, according to one example embodiment. In this embodiment, the motion sensing device 104 includes sensors 202, a communication module 208, and firmware 210. However additional or different sensors 202 may be included in the motion sensing device 104. In one embodiment the user places the motion sensing device 104 on their body, such as on their arm, wrist, leg or ankle. In another embodiment, the user places the motion sensing device on the fitness system, such as on the handle bars of an elliptical machine. The sensors 202 generate motion data based on an activity performed by the user. The activity performed by the user may include, running, cycling, using the elliptical machine, or any other form of exercise. In one embodiment the sensors 202 may include an accelerometer 204 and a gyroscope 206. In one embodiment, the accelerometer 204 captures acceleration data 204 based on the acceleration of the motion sensing device 104 along multiple axes. In one embodiment, the gyroscope 206 captures a rate of rotation of the motion sensing device 104 along multiple axes. In one embodiment, the firmware 210 in the motion sensing device 104 is a combination of a persistent memory, a program stored in the memory and a set of hardware required to execute the program. The firmware 210 retrieves, formats and temporarily stores the data output by the accelerometer 204 and the gyroscope 206. In other embodiments the firmware 210 may perform other activities, such as controlling the rate at which data is retrieved from the accelerometer 204 and the gyroscope 206 or averaging samples of data retrieved from the accelerometer 204 and the gyroscope 206. In another embodiment, the firmware 210 contains logic that manages the operation of the motion sensing device 104. For example, the firmware 210 may contain logic to support a “shake to connect” function that monitors the sensors 202 and establishes the communication link 108A to the host device 110 upon detecting that a user has shaken the motion sensing device 104. In an alternative embodiment described with reference to FIG. 34, the two hand controllers 106A, 106B are coupled to the motion sensing device 104 and the motion sensing device 104 is configured to relay input data from the hand controllers 106A, 106B to the host device 110. In this alternative embodiment, the firmware 210 is configured to detect and connect to the hand controllers 106A, 106B independently of the host device 110, and the communication module 208 maintains the connections with the hand controllers 106A, 106B after they are established. The connections with the hand controllers 106A, 106B may be under the same communication protocol as the communication link 108A (e.g., Bluetooth Low Energy) or a different communication protocol (e.g., an RF connection). Meanwhile, the firmware 210 manages the three connections (the connections with the two hand controllers 106A, 106B and the communication link 108A with the host device 110) and relays input data from the hand controllers 106A, 106B to the host device 110. In some embodiments, the firmware 210 may be configured to combine the input data from the sensors 202 and the hand controllers 106A, 106B into a single integrated input stream. Thus, the host device API 112 receives a single input stream that includes inputs from all three hardware devices. In one embodiment, the communication module 208 transmits the formatted acceleration and rate of rotation data from the firmware 210 to the host device 110. The communication module 208 interacts with the communication link 108A to transfer data between the motion sensing device 104 and the host device 110. In one embodiment, the communication module 208 connects via the Bluetooth Low Energy (BLE) protocol.

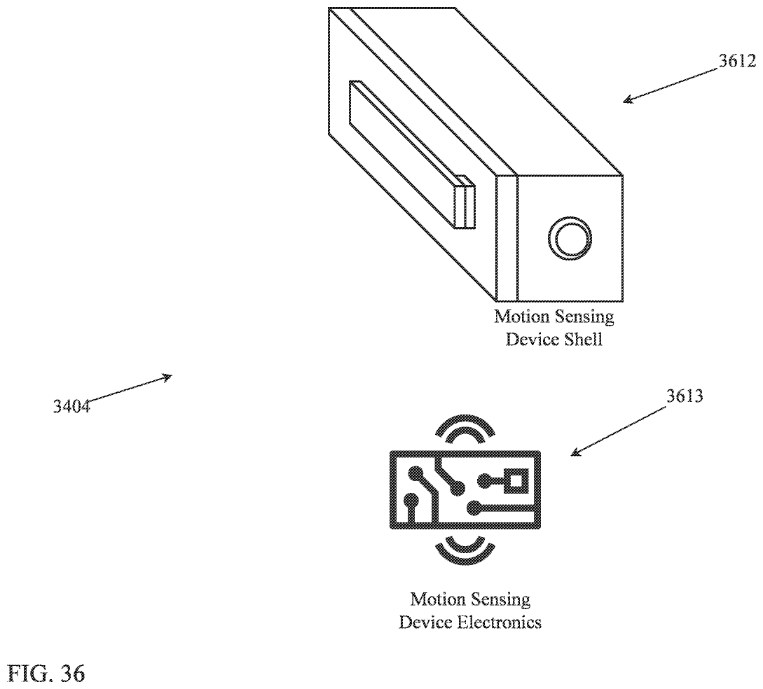

FIG. 36 illustrates a motion sensing device 104, according to another example embodiment. In this embodiment, the motion sensing device 104 is comprised of an electronic circuit 213, and an outer shell 212. The outer shell 212 houses the electronic circuit 213. The outer shell also contains an attachment mechanism to attach the motion sensing device 104 to the user or the fitness system. For example, the user can attach the motion sensing device 104 to their wrist, ankle or to the handle bars on an elliptical machine via the attachment mechanism. The electronic circuit is comprised of the accelerometer 204, the gyroscope 206, the communication module 208 and firmware 210. In the illustrated embodiment, the motion sensing device 104 connects wirelessly to the host device 110 and is powered by a battery.

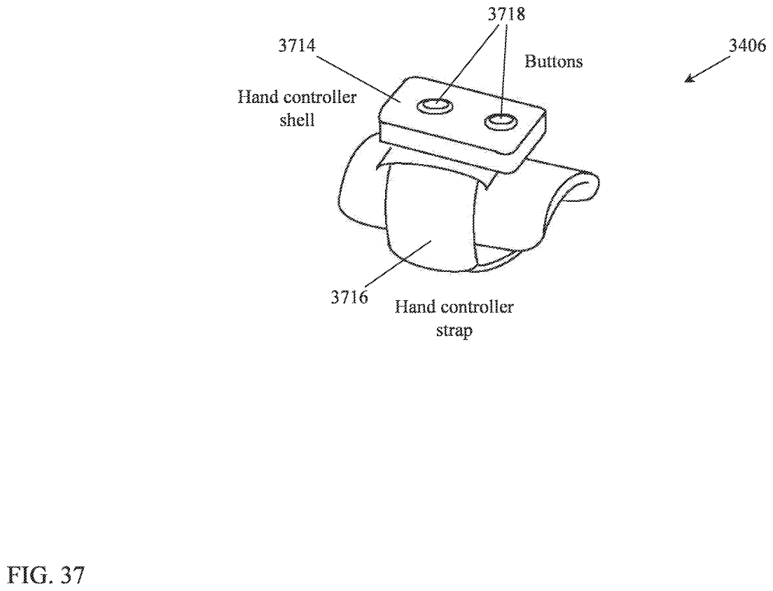

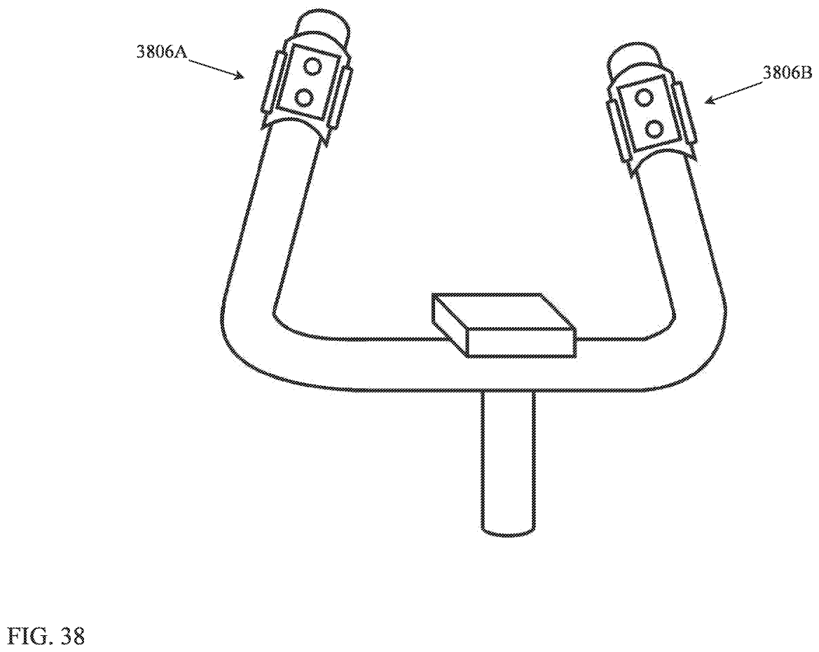

FIG. 37 illustrates a hand controller 106, according to one example embodiment. In this embodiment the hand controller 106 is comprised of an attaching mechanism 216, a hand controller shell 214 and buttons 218. Apart from buttons 218 the hand controller may also include a directional pad or joystick either in addition to or in place of the buttons 218. As described above with reference to FIG. 34, the hand controller 106 communicates with the host device 110. In the illustrated embodiment, the hand controller 106 communicates wirelessly and is battery powered. In one embodiment, the attaching mechanism 216 is used to attach the hand controller to the fitness system or the user. For example, the attaching mechanism 216 is a strap used to strap the hand controller 106 onto the handle bar of an elliptical machine. In other embodiments, the attaching mechanism is not limited to a strap. In one embodiment, the hand controller shell 214 houses the electronic components of the hand controller 106 and other relevant components such as a battery. The form factor of the hand controller shell 214 facilitates the attaching of the hand controller 106 to the user or the fitness system. In one embodiment, the buttons 218 on the hand controller 106 receive user input. Internally, the hand controller 106 includes a radio and a microcontroller. In one embodiment, the radio is an RF radio that connects to the motion sensing device 104 and sends an input signal to the motion sensing device 104 to be relayed to the host device 110. In an alternative embodiment, the hand controller includes a Bluetooth Low Energy (BLE) radio and a system on a chip (SoC). In some embodiments, the radio, microcontroller, and any other electronic components of the hand controller 106 may be placed on a single printed circuit board.

FIG. 38 illustrates an example of hand controllers 106A, 106B mounted to handlebars of a fitness device, according to one embodiment. The straps 216 of the hand controllers 106A, 106B are wrapped around the handlebars. In one embodiment, the bottom side of the hand controller shell 214 may include an adhesive substance to increase the friction between the hand controllers 106A, 106B and the handlebars. To reduce manufacturing costs, some or all of the components of the two hand controllers 106A, 106B may be identical. In one embodiment, the two hand controllers 106A, 106B are completely identical (e.g., same mechanical enclosure, internal electronics, and firmware). In this embodiment, it may be difficult for the firmware 210 or the host device API 112 to distinguish between the two hand controllers, and the interface software 111 may prompt the user to identify each hand controller as described above with reference to FIG. 34. In another embodiment, the two hand controllers have the same internal electronics (e.g., the same microcontroller, radio, etc.), but may be placed in different enclosures to identify one controller as the left hand controller 106A and the other as the right hand controller 106B. For example, the buttons 218 on the two hand controllers may have a different layout or different colors, or the housings may have symmetric ergonomic features so that they are better suited for a user's hands. The two hand controllers 106 may also be flashed with different firmware so that the motion sensing device 104 or host device API 112 can distinguish between the input data from the two controllers 106. For example, the firmware on the left hand controller 106A may be configured to send an identifier (e.g., a binary 0) in a header of its input stream, while the firmware on the second hand controller 106B may be configured to send a different identifier (e.g., a binary 1) in a header of its input stream.

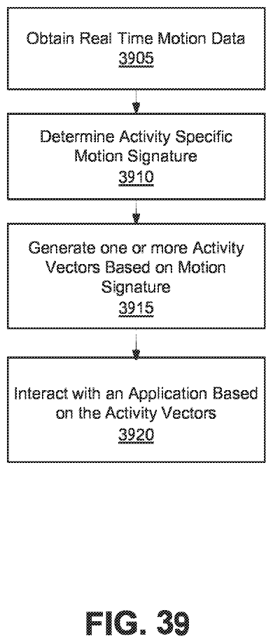

FIG. 39 is a flowchart illustrating an example process for interacting with a fitness application 114 based on the user's interaction with the fitness system, according to one embodiment. The user begins by attaching the hardware devices 102 to a fitness device or to the user's body; the hand controllers 106A, 106B can either be held in the user's hands or attached to the fitness device, and the motion sensing device 104 may be attached to the user's body or to the fitness device, depending on which fitness application 114 the user selects. One of the fitness applications 114 may be an instruction application that displays instructions for mounting the hardware devices 102 on a selected fitness device. In one embodiment, the motion sensing device 104 obtains 305 real time motion data comprised of acceleration data from the accelerometer 204 and rotation data from the gyroscope 206, as the user interacts with the fitness system. The fitness system is not limited to a stationary exercise machine such as a treadmill, but may also include dynamic means of exercise, such as running on the road. The data obtained by the motion sensing device is 104 is transmitted to the host device 110 over established communication links 108. Based on the data obtained 305, the interface software 111 on the host device determine 310 an activity specific motion signature. The activity specific motion signature identifies the current activity performed by the user. In one embodiment, the motion signature acts as a basis against which future movements performed by the user are compared against by the fitness applications 114. In one embodiment, the interface software 111 generates one or more activity vectors 315 based on the motion signature. The activity vectors represent metrics associated with the activity level of the user. For example, the activity vectors may represent the number of calories burned by a user, the distance covered over a specific time interval or the intensity level of the user. The activity vectors are a standard unit of measurement across different types of fitness devices. In one embodiment, the user interacts 320 with the fitness application 114 based on the activity vector generated 315 by the interface software 111. By altering the intensity with which a user performs a fitness activity, the user changes the activity vector over a period of time. The change in the activity vector alters the state of an object in the fitness application 114, or the behavior of the object in the fitness application 114 thereby allowing the user to interact with the fitness application 114 on the host device 110. For example, if the user is playing a running video game, where the user's character is running on the screen through an obstacle course, the intensity at which the user runs on the treadmill or cycles on a bicycle influences the speed at which the user's character (the object in an application 114) runs across the screen (behavior exhibited by the object) in the video game. Thus by running faster, the user changes the activity vector in real time, corresponding to an increase in speed of the user's character. The user may also interact with the application based on input from the hand controllers 106. For example, the user could press a button on the hand controller 106 as the user's character approaches an obstacle in the video game, causing the user's character to jump over or avoid the obstacle.

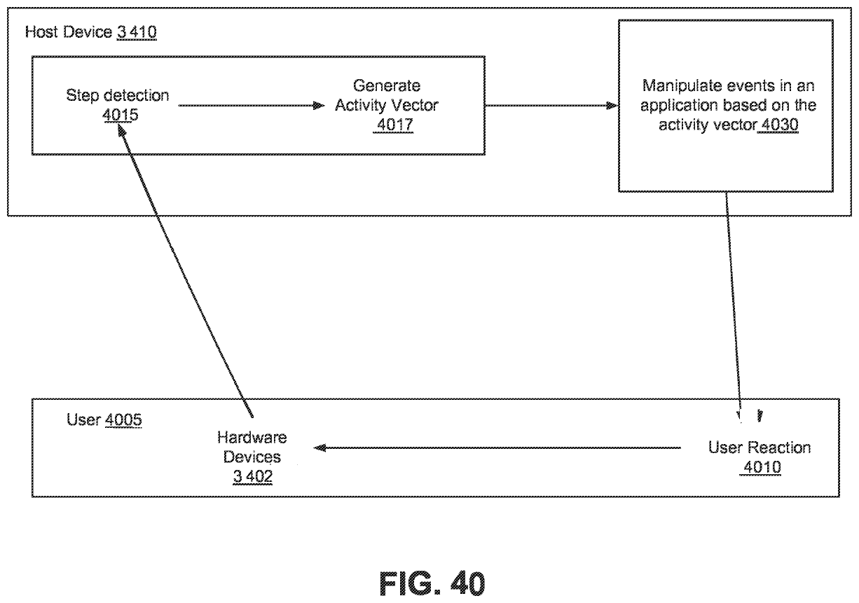

FIG. 40 illustrates an example process of a user 405 manipulating events in a fitness application 114 based on the user's 405 interaction with the fitness system in real time, according to one embodiment. In this embodiment, the events in a fitness application 114 may influence a user reaction 410. The user reaction 410 is any change in user activity or intensity of the fitness activity performed by the user 405. For example, the user 405 may notice that the user's character in a video game is not running fast enough to avoid a disaster. This may generate the user reaction 410, where the user 405 begins to run faster while running on a treadmill. In other, embodiments the user 405 may make no change to the intensity at which the user 405 performs a fitness activity. The hardware devices 102 capture motion data and input data to the hand controllers as the user 405 performs the fitness activity. In one embodiment, the user reaction 410 generates an increase in the magnitude of the motion data captured by the hardware devices 102, as compared to previous motion data captured by the hardware devices 102. The hardware devices 102 transmit the captured data to the host device 110 over communication links 108. The interface software 111 on the host device 110 processes the motion data captured by the hardware devices 102. In one embodiment, the interface software 111, executes a step detection 415 function, to determine the steps taken by a user 405 over a time period. The step detection is performed using the activity specific motion signature 310 determined by the interface software 111, as a basis. In one embodiment, a step represents a unit of movement of the fitness activity performed by the user 405. For example, a single step while walking and running, or a single rotation of a bicycle pedal while cycling would be detected 415 as a single step by the interface software 111. In one embodiment, the interface software 111 generates an activity vector 345 based on the number of steps over a time window, or a time span over which a single step takes place. The motion signature of the activity performed by the user 405, determines how the number of steps over a specific time window, or the time between two steps influences the value of the activity vector. For example, one revolution on a bicycle may be considered as 2 steps while running. Thus, the interface software 111, identifies what activity a step is related to based on the motion signature determined by the interface software 111. Then the interface software 111 modifies or generates the activity vector based on the number of steps in a given time window, or the time between steps and the motion signature associated with the step. This allows the user 405 to switch between fitness machines, such as move from running on a treadmill to cycling on a fitness bicycle, but still generate consistent activity vectors representing the intensity at which the user 405 performs different fitness activities across different fitness machines. In one embodiment, the number of steps within a given time window represents the motion signature associated with a fitness activity. For example, running may correspond to fewer steps being detected in the given time window as compared to bicycling. In another embodiment, the activity vector is an intensity level that is scaled inversely to the time between steps for a specific motion signature. For example, if the time between steps is 0.5 seconds the intensity level is given a value of 5 (on a scale of 0 to 10). However, when the time between steps reduces to 0.4, the intensity level is given a value of 7 (on a scale of 0 to 10). In a different embodiment, the activity vector is an intensity level inversely to the number of steps in a given time window. Based on the activity vector the user 405 manipulates events in the fitness application 114 in real time. In one embodiment, elements or objects in the fitness application 114 are given attributes based on the activity vector generated 315 by the interface software 111. For example, the fitness application 114 may be a game, where the rate at which points are earned by the user 405 increases as the magnitude of the activity vector increases. Hence, the user 405 may run faster on a treadmill to increase the number of steps per given time window, thereby increasing the magnitude of the generated activity vector, manipulating 430 the rate at which points are increased in the game.

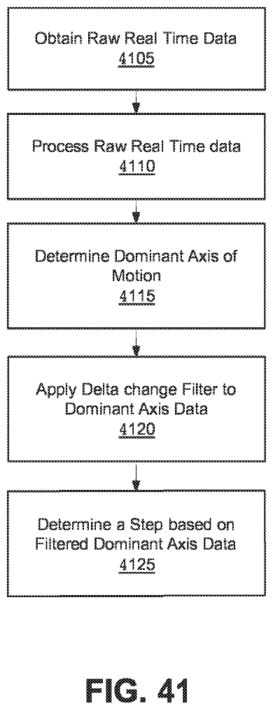

FIG. 41 is a flow chart illustrating an example process for determining a step taken by the user, based on motion data, according to one embodiment. The hardware devices 102 obtain 505 raw real time motion data as the user performs the fitness activity. The hardware devices 102 transmit the raw motion data to the host device 110 and interface software 111 over communication links 108. In one embodiment, the interface software 111 processes 510 the raw motion data. There are a number of techniques that may be applied to process 510 the raw motion data. For example, the interface software may use a high pass filter 510 to process the raw motion data on each axis of motion or rotation. The high pass filter attenuates or reduces the magnitude of data with frequencies below a cutoff frequency. In one embodiment, the interface software 111 determines 515 a dominant axis of the processed motion data. The dominant axis represents the axis along which there is most motion captured by the hardware devices 102, as the user performs the fitness activity. Determining 515 the dominant axis of motion, helps the interface software 111 identify the primary direction or axis of motion of the user as the user performs a fitness activity. In one embodiment, the primary direction of motion of the user influences at least in part the motion signature generated by the interface software 111. In one embodiment, the dominant axis is determined 515 by summing the absolute value of each axis of processed motion data over a rolling time window. In one embodiment, the axis with the largest absolute value over the rolling time window is picked as the dominant axis of motion. If a good set of data is not obtained for the current dominant axis, the dominant axis is changed to the axis with the next largest absolute value. In one embodiment, a good set of data is determined by comparing the ratio of the absolute value of the current dominant axis to the sum of the absolute values of the other axes, and a threshold value. In one embodiment, a delta change filter is applied 520 to the dominant axis of the processed 510 motion data. The delta change filter increases or decreases the difference between a current value and in a time series of motion data and a previous value in the time series. For example, if the current value of the processed motion data is similar to the previous value in the time series, the delta change filter changes the current value in the time series to equal that of the previous value. If the current value is significantly different from the previous value the delta change filter changes the current value by an interpolated amount so that the difference between the current value and the previous value increases (i.e., the absolute difference). In one embodiment, the interface software 111 determines 525 the step taken by the user based on the time series of the processed data along the dominant axis of motion. For example, a step is detected 525 when there is a sign change in the acceleration data of the determined 520 dominant axis. In another embodiment, an acceleration threshold must be crossed just before the sign change in the acceleration data, for the step to be considered a potential step. For example, the time series acceleration value must be greater than 0.5G, before a sign change is identified in a time window containing 20 accelerometer samples collected over 333 milliseconds (ms).

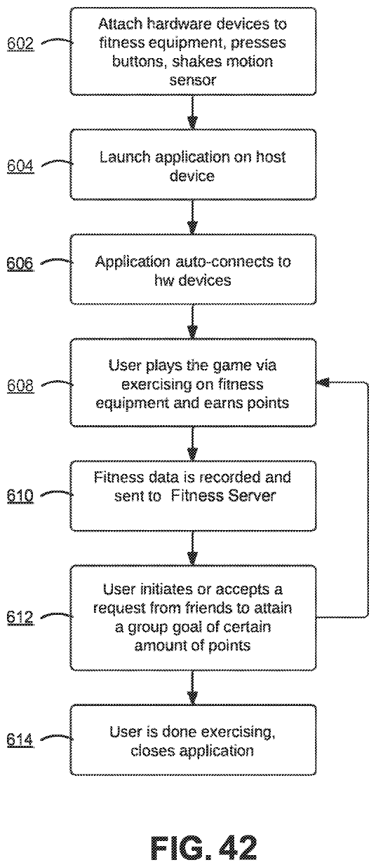

FIG. 42 is a flow chart illustrating an example process for interacting with the adaptable fitness system, according to one embodiment. The user begins by attaching 602 the hardware devices 102 to a fitness device or to the user's body; the hand controllers 106A, 106B can either be held in the user's hands or attached to the fitness device, and the motion sensing device 104 may be attached to the user's body or to the fitness device, depending on which fitness application 114 the user selects. One of the fitness applications 114 may be an instruction application that displays instructions for mounting the hardware devices 102 on a selected fitness device. Next, the user selects and launches 604 a fitness application 114, and the host device API 112 initiates a search to auto-connect 606 with the hardware devices 102, which runs in the background. The application 114 provides a user interface indicator showing the state in which the hardware devices 102 are connecting. The user then begins to interact 608 with the application 114. As the user works out, the motion sensing device 104 sends input data to the application 114 tracking player movement. The hand controllers 106A, 106B send input data to the application tracking player game inputs. The application 114 shows the user game cues and feedback. The operating system of the host device 110 runs the application. The host device 110 records 610 fitness data for the user to review. The fitness data is reflected in quantifiers such as steps taken and calories burned. The user is encouraged to exercise by seeing his game progression and fitness data. The application 114 may optionally use the host device API 112 to send the fitness data to the fitness server 118 to be saved. If the host device 110 does not have network connectivity at the time the fitness data is recorded 610, then the data may be automatically sent when network connectivity becomes available. If the user chooses to use the fitness application 114 anonymously (i.e., without logging in to a user account), then the user's session is tracked anonymously. The user is also able to share his game progression and fitness data online with friends on social networking systems, which provides further encouragement. In addition, the user may use social features of the fitness server 118, such as initiating/accepting requests 612 to attain a group goal (e.g., a specified number of movement units) or initiating/accepting challenges to attain an individual goal. If a request or challenge is activated, then the user begins interacting 508 with the application 114 again. When the user is done, the user ends the session by closing 614 the application 114. In one embodiment, the user can create a personal goal to achieve an activity vector value in a given time period. For example, a goal can be to run 50 miles in one week, while maintaining an intensity level of 7 (based on an intensity level scale ranging from 0 to 10). These personal goals can then be shared with friends within a social network. Users can create also cooperative goals for a group in a social network to achieve an activity vector value. For example, a group of five people may set a goal to achieve 250 miles for the week, while all maintaining the intensity level of 7. Activity vectors can also be used as a basis for activities that benefit from normalization of data for comparison purposes. For example, activity vectors can be used in competitions between groups or friends in a network. Challenges can be issued and broadcast to a user's friends on social network.

### Exemplary Computing Environment

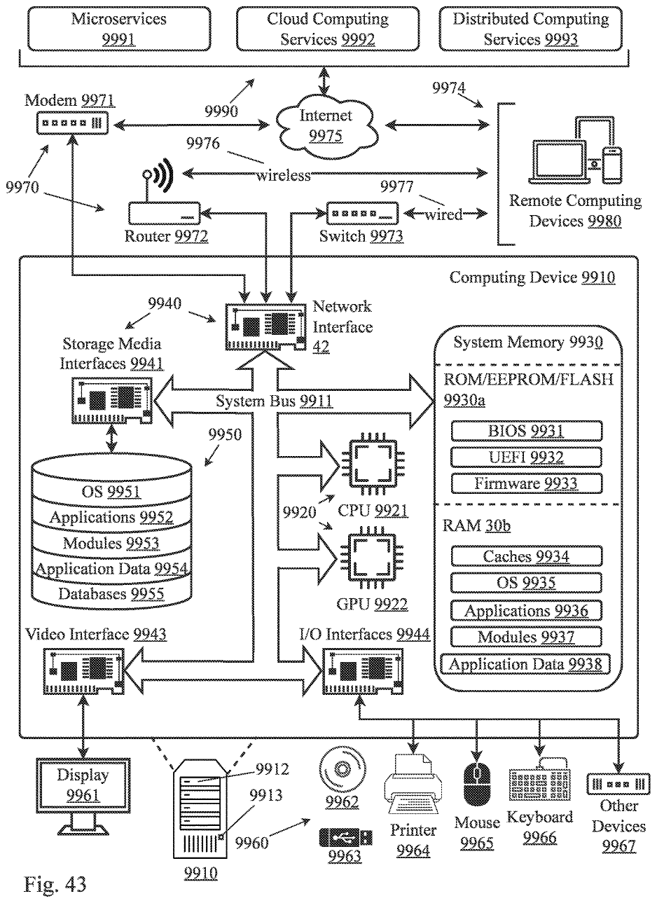

FIG. 43 illustrates an exemplary computing environment on which an embodiment described herein may be implemented, in full or in part. This exemplary computing environment describes computer-related components and processes supporting enabling disclosure of computer-implemented embodiments. Inclusion in this exemplary computing environment of well-known processes and computer components, if any, is not a suggestion or admission that any embodiment is no more than an aggregation of such processes or components. Rather, implementation of an embodiment using processes and components described in this exemplary computing environment will involve programming or configuration of such processes and components resulting in a machine specially programmed or configured for such implementation. The exemplary computing environment described herein is only one example of such an environment and other configurations of the components and processes are possible, including other relationships between and among components, and/or absence of some processes or components described. Further, the exemplary computing environment described herein is not intended to suggest any limitation as to the scope of use or functionality of any embodiment implemented, in whole or in part, on components or processes described herein.

The exemplary computing environment described herein comprises a computing device 9910 (further comprising a system bus 9911, one or more processors 9920, a system memory 9930, one or more interfaces 9940, one or more non-volatile data storage devices 9950), external peripherals and accessories 9960, external communication devices 9970, remote computing devices 9980, and cloud-based services 9990.

System bus 9911 couples the various system components, coordinating operation of and data transmission between, those various system components. System bus 9911 represents one or more of any type or combination of types of wired or wireless bus structures including, but not limited to, memory busses or memory controllers, point-to-point connections, switching fabrics, peripheral busses, accelerated graphics ports, and local busses using any of a variety of bus architectures. By way of example, such architectures include, but are not limited to, Industry Standard Architecture (ISA) busses, Micro Channel Architecture (MCA) busses, Enhanced ISA (EISA) busses, Video Electronics Standards Association (VESA) local busses, a Peripheral Component Interconnects (PCI) busses also known as a Mezzanine busses, or any selection of, or combination of, such busses. Depending on the specific physical implementation, one or more of the processors 9920, system memory 9930 and other components of the computing device 9910 can be physically co-located or integrated into a single physical component, such as on a single chip. In such a case, some or all of system bus 9911 can be electrical pathways within a single chip structure.

Computing device may further comprise externally-accessible data input and storage devices 9912 such as compact disc read-only memory (CD-ROM) drives, digital versatile discs (DVD), or other optical disc storage for reading and/or writing optical discs 9962; magnetic cassettes, magnetic tape, magnetic disk storage, or other magnetic storage devices; or any other medium which can be used to store the desired content and which can be accessed by the computing device 9910. Computing device may further comprise externally-accessible data ports or connections 9912 such as serial ports, parallel ports, universal serial bus (USB) ports, and infrared ports and/or transmitter/receivers. Computing device may further comprise hardware for wireless communication with external devices such as IEEE 1394 (“Firewire”) interfaces, IEEE 802.11 wireless interfaces, BLUETOOTH® wireless interfaces, and so forth. Such ports and interfaces may be used to connect any number of external peripherals and accessories 9960 such as visual displays, monitors, and touch-sensitive screens 9961, USB solid state memory data storage drives (commonly known as “flash drives” or “thumb drives”) 9963, printers 9964, pointers and manipulators such as mice 9965, keyboards 9966, and other devices 9967 such as joysticks and gaming pads, touchpads, additional displays and monitors, and external hard drives (whether solid state or disc-based), microphones, speakers, cameras, and optical scanners.

Processors 9920 are logic circuitry capable of receiving programming instructions and processing (or executing) those instructions to perform computer operations such as retrieving data, storing data, and performing mathematical calculations. Processors 9920 are not limited by the materials from which they are formed or the processing mechanisms employed therein, but are typically comprised of semiconductor materials into which many transistors are formed together into logic gates on a chip (i.e., an integrated circuit or IC). The term processor includes any device capable of receiving and processing instructions including, but not limited to, processors operating on the basis of quantum computing, optical computing, mechanical computing (e.g., using nanotechnology entities to transfer data), and so forth. Depending on configuration, computing device 9910 may comprise more than one processor. For example, computing device 9910 may comprise one or more central processing units (CPUs) 9921, each of which itself has multiple processors or multiple processing cores, each capable of independently or semi-independently processing programming instructions. Further, computing device 9910 may comprise one or more specialized processors such as a graphics processing unit (GPU) 9922 configured to accelerate processing of computer graphics and images via a large array of specialized processing cores arranged in parallel.

System memory 9930 is processor-accessible data storage in the form of volatile and/or nonvolatile memory. System memory 9930 may be either or both of two types: non-volatile memory and volatile memory. Non-volatile memory 9930a is not erased when power to the memory is removed, and includes memory types such as read only memory (ROM), electronically-erasable programmable memory (EEPROM), and rewritable solid state memory (commonly known as “flash memory”). Non-volatile memory 9930a is typically used for long-term storage of a basic input/output system (BIOS) 9931, containing the basic instructions, typically loaded during computer startup, for transfer of information between components within computing device, or a unified extensible firmware interface (UEFI), which is a modern replacement for BIOS that supports larger hard drives, faster boot times, more security features, and provides native support for graphics and mouse cursors. Non-volatile memory 9930a may also be used to store firmware comprising a complete operating system 9935 and applications 9936 for operating computer-controlled devices. The firmware approach is often used for purpose-specific computer-controlled devices such as appliances and Internet-of-Things (IoT) devices where processing power and data storage space is limited. Volatile memory 9930b is erased when power to the memory is removed and is typically used for short-term storage of data for processing. Volatile memory 9930b includes memory types such as random access memory (RAM), and is normally the primary operating memory into which the operating system 9935, applications 9936, program modules 9937, and application data 9938 are loaded for execution by processors 9920. Volatile memory 9930b is generally faster than non-volatile memory 9930a due to its electrical characteristics and is directly accessible to processors 9920 for processing of instructions and data storage and retrieval. Volatile memory 9930b may comprise one or more smaller cache memories which operate at a higher clock speed and are typically placed on the same IC as the processors to improve performance.

Interfaces 9940 may include, but are not limited to, storage media interfaces 9941, network interfaces 9942, display interfaces 9943, and input/output interfaces 9944. Storage media interface 9941 provides the necessary hardware interface for loading data from non-volatile data storage devices 9950 into system memory 9930 and storage data from system memory 9930 to non-volatile data storage device 9950. Network interface 9942 provides the necessary hardware interface for computing device 9910 to communicate with remote computing devices 9980 and cloud-based services 9990 via one or more external communication devices 9970. Display interface 9943 allows for connection of displays 9961, monitors, touchscreens, and other visual input/output devices. Display interface 9943 may include a graphics card for processing graphics-intensive calculations and for handling demanding display requirements. Typically, a graphics card includes a graphics processing unit (GPU) and video RAM (VRAM) to accelerate display of graphics. One or more input/output (I/O) interfaces 9944 provide the necessary support for communications between computing device 9910 and any external peripherals and accessories 9960. For wireless communications, the necessary radio-frequency hardware and firmware may be connected to I/O interface 9944 or may be integrated into I/O interface 9944.

Non-volatile data storage devices 9950 are typically used for long-term storage of data. Data on non-volatile data storage devices 9950 is not erased when power to the non-volatile data storage devices 9950 is removed. Non-volatile data storage devices 9950 may be implemented using any technology for non-volatile storage of content including, but not limited to, CD-ROM drives, digital versatile discs (DVD), or other optical disc storage; magnetic cassettes, magnetic tape, magnetic disc storage, or other magnetic storage devices; solid state memory technologies such as EEPROM or flash memory; or other memory technology or any other medium which can be used to store data without requiring power to retain the data after it is written. Non-volatile data storage devices 9950 may be non-removable from computing device 9910 as in the case of internal hard drives, removable from computing device 9910 as in the case of external USB hard drives, or a combination thereof, but computing device will typically comprise one or more internal, non-removable hard drives using either magnetic disc or solid state memory technology. Non-volatile data storage devices 9950 may store any type of data including, but not limited to, an operating system 9951 for providing low-level and mid-level functionality of computing device 9910, applications 9952 for providing high-level functionality of computing device 9910, program modules 9953 such as containerized programs or applications, or other modular content or modular programming, application data 9954, and databases 9955 such as relational databases, non-relational databases, and graph databases.

Applications (also known as computer software or software applications) are sets of programming instructions designed to perform specific tasks or provide specific functionality on a computer or other computing devices. Applications are typically written in high-level programming languages such as C++, Java, and Python, which are then either interpreted at runtime or compiled into low-level, binary, processor-executable instructions operable on processors 9920. Applications may be containerized so that they can be run on any computer hardware running any known operating system. Containerization of computer software is a method of packaging and deploying applications along with their operating system dependencies into self-contained, isolated units known as containers. Containers provide a lightweight and consistent runtime environment that allows applications to run reliably across different computing environments, such as development, testing, and production systems.

The memories and non-volatile data storage devices described herein do not include communication media. Communication media are means of transmission of information such as modulated electromagnetic waves or modulated data signals configured to transmit, not store, information. By way of example, and not limitation, communication media includes wired communications such as sound signals transmitted to a speaker via a speaker wire, and wireless communications such as acoustic waves, radio frequency (RF) transmissions, infrared emissions, and other wireless media.

External communication devices 9970 are devices that facilitate communications between computing device and either remote computing devices 9980, or cloud-based services 9990, or both. External communication devices 9970 include, but are not limited to, data modems 9971 which facilitate data transmission between computing device and the Internet 9975 via a common carrier such as a telephone company or internet service provider (ISP), routers 9972 which facilitate data transmission between computing device and other devices, and switches 9973 which provide direct data communications between devices on a network. Here, modem 9971 is shown connecting computing device 9910 to both remote computing devices 9980 and cloud-based services 9990 via the Internet 9975. While modem 9971, router 9972, and switch 9973 are shown here as being connected to network interface 9942, many different network configurations using external communication devices 9970 are possible. Using external communication devices 9970, networks may be configured as local area networks (LANs) for a single location, building, or campus, wide area networks (WANs) comprising data networks that extend over a larger geographical area, and virtual private networks (VPNs) which can be of any size but connect computers via encrypted communications over public networks such as the Internet 9975. As just one exemplary network configuration, network interface 9942 may be connected to switch 9973 which is connected to router 9972 which is connected to modem 9971 which provides access for computing device 9910 to the Internet 9975. Further, any combination of wired 9977 or wireless 9976 communications between and among computing device 9910, external communication devices 9970, remote computing devices 9980, and cloud-based services 9990 may be used. Remote computing devices 9980, for example, may communicate with computing device through a variety of communication channels 9974 such as through switch 9973 via a wired 9977 connection, through router 9972 via a wireless connection 9976, or through modem 9971 via the Internet 9975. Furthermore, while not shown here, other hardware that is specifically designed for servers may be employed. For example, secure socket layer (SSL) acceleration cards can be used to offload SSL encryption computations, and transmission control protocol/internet protocol (TCP/IP) offload hardware and/or packet classifiers on network interfaces 9942 may be installed and used at server devices.

In a networked environment, certain components of computing device 9910 may be fully or partially implemented on remote computing devices 9980 or cloud-based services 9990. Data stored in non-volatile data storage device 9950 may be received from, shared with, duplicated on, or offloaded to a non-volatile data storage device on one or more remote computing devices 9980 or in a cloud computing service 9992. Processing by processors 9920 may be received from, shared with, duplicated on, or offloaded to processors of one or more remote computing devices 9980 or in a distributed computing service 9993. By way of example, data may reside on a cloud computing service 9992, but may be usable or otherwise accessible for use by computing device 9910. Also, certain processing subtasks may be sent to a microservice 9991 for processing with the result being transmitted to computing device 9910 for incorporation into a larger processing task. Also, while components and processes of the exemplary computing environment are illustrated herein as discrete units (e.g., OS 9951 being stored on non-volatile data storage device 9951 and loaded into system memory 9935 for use) such processes and components may reside or be processed at various times in different components of computing device 9910, remote computing devices 9980, and/or cloud-based services 9990.

Remote computing devices 9980 are any computing devices not part of computing device 9910. Remote computing devices 9980 include, but are not limited to, personal computers, server computers, thin clients, thick clients, personal digital assistants (PDAs), mobile telephones, watches, tablet computers, laptop computers, multiprocessor systems, microprocessor based systems, set-top boxes, programmable consumer electronics, video game machines, game consoles, portable or handheld gaming units, network terminals, desktop personal computers (PCs), minicomputers, main frame computers, network nodes, and distributed or multi-processing computing environments. While remote computing devices 9980 are shown for clarity as being separate from cloud-based services 9990, cloud-based services 9990 are implemented on collections of networked remote computing devices 9980.

Cloud-based services 9990 are Internet-accessible services implemented on collections of networked remote computing devices 9980. Cloud-based services are typically accessed via application programming interfaces (APIs) which are software interfaces which provide access to computing services within the cloud-based service via API calls, which are pre-defined protocols for requesting a computing service and receiving the results of that computing service. While cloud-based services may comprise any type of computer processing or storage, three common categories of cloud-based services 9990 are microservices 9991, cloud computing services 9992, and distributed computing services 9993.

Microservices 9991 are collections of small, loosely coupled, and independently deployable computing services. Each microservice represents a specific computing functionality and runs as a separate process or container. Microservices promote the decomposition of complex applications into smaller, manageable services that can be developed, deployed, and scaled independently. These services communicate with each other through well-defined application programming interfaces (APIs), typically using lightweight protocols like HTTP or message queues. Microservices 9991 can be combined to perform more complex processing tasks.

Cloud computing services 9992 are delivery of computing resources and services over the Internet 9975 from a remote location. Cloud computing services 9992 provide additional computer hardware and storage on as-needed or subscription basis. Cloud computing services 9992 can provide large amounts of scalable data storage, access to sophisticated software and powerful server-based processing, or entire computing infrastructures and platforms. For example, cloud computing services can provide virtualized computing resources such as virtual machines, storage, and networks, platforms for developing, running, and managing applications without the complexity of infrastructure management, and complete software applications over the Internet on a subscription basis.

Distributed computing services 9993 provide large-scale processing using multiple interconnected computers or nodes to solve computational problems or perform tasks collectively. In distributed computing, the processing and storage capabilities of multiple machines are leveraged to work together as a unified system. Distributed computing services are designed to address problems that cannot be efficiently solved by a single computer or that require large-scale computational power. These services enable parallel processing, fault tolerance, and scalability by distributing tasks across multiple nodes.

Although described above as a physical device, computing device 9910 can be a virtual computing device, in which case the functionality of the physical components herein described, such as processors 9920, system memory 9930, network interfaces 9940, and other like components can be provided by computer-executable instructions. Such computer-executable instructions can execute on a single physical computing device, or can be distributed across multiple physical computing devices, including being distributed across multiple physical computing devices in a dynamic manner such that the specific, physical computing devices hosting such computer-executable instructions can dynamically change over time depending upon need and availability. In the situation where computing device 9910 is a virtualized device, the underlying physical computing devices hosting such a virtualized computing device can, themselves, comprise physical components analogous to those described above, and operating in a like manner. Furthermore, virtual computing devices can be utilized in multiple layers with one virtual computing device executing within the construct of another virtual computing device. Thus, computing device 9910 may be either a physical computing device or a virtualized computing device within which computer-executable instructions can be executed in a manner consistent with their execution by a physical computing device. Similarly, terms referring to physical components of the computing device, as utilized herein, mean either those physical components or virtualizations thereof performing the same or equivalent functions.

The skilled person will be aware of a range of possible modifications of the various aspects described above. Accordingly, the present invention is defined by the claims and their equivalents.

What is claimed is:

1. An exercise device with computer gaming system integration wherein the exercise device acts as a gaming controller for computer gaming systems, comprising:
a frame;
a first and a second crank system mounted opposing sides of the frame adapted for positioning a user therebetween, each of the first and second crank system comprising a first crank assembly rotatable about a first axis and a second crank assembly rotatable about a second axis with the first and second axes each being displaced from the other, each of the first crank assemblies comprising a first crank arm, and each of the second crank assemblies comprising a second crank arm;
a pair of synchronous control system assemblies one mechanically coupled to the first crank system and the other mechanically coupled to the second crank system, each synchronous control system assembly of the pair of synchronous control system assemblies being mechanically coupled to the first and second crank assemblies of the corresponding crank system in a configuration that allows for 360 degrees of synchronous rotation of the first and second crank assemblies, wherein each first crank arm of each first crank assembly rotates in the same direction as the second crank arm of each second crank assembly by the corresponding synchronous control system assembly of the pair of synchronous control system assemblies;
a pair of pedal arms, one coupled to the first crank system and the other coupled to the second crank system, each of the pair of pedal arms being pivotally coupled at a first end to the first crank arm of the first crank assembly and being pivotally coupled at a second end to a pedal;
a pair of movable crank links, one corresponding to each of the first and second crank systems, each of the pair of movable crank links comprising a first attachment point, a second attachment point, and a flexible attachment between the first attachment point and second attachment point, wherein the first attachment point of each movable crank link is pivotally coupled to the pedal arm of its corresponding crank system and the second attachment point of each movable crank is pivotally coupled to the second crank arm of its corresponding second crank assembly; and
a sensor configured to measure a movement of a component of the exercise device and transmit the measurement as data to a compatible computing device.

2. The system of claim 1, wherein the exercise device further comprises a game controller, wherein:
the game controller comprises a computer interface for communicating with the compatible computing device; and
the game controller is configured to transmit game control data to the compatible computing device via the computer interface.

3. The system of claim 2, wherein the game controller comprises buttons or a touchscreen for game input by the user.

4. The system of claim 1, wherein the exercise device further comprises a pair of game controllers one on each of two sides of the exercise device and configured to transmit game control data to the compatible computing device, wherein each game controller is accessible to a hand of the user.

5. The system of claim 1, wherein the sensor is configured to continually or periodically measure a distance between the first attachment point and second attachment point.

6. The system of claim 5, wherein the sensor is configured to calculate a stride length and stride period based on the continually or periodically measured distance.

7. The system of claim 1, wherein the sensor is configured to measure a speed of operation of one of the pair of synchronous control systems.

8. The system of claim 7, wherein the sensor is configured to calculate a power output of the user based on the measured speed of operation.

9. The system of claim 2, wherein the sensor is configured to transmit data to the compatible computing device through the computer interface of the game controller.

10. The system of claim 4, wherein the sensor is configured to transmit data to the compatible computing device through the computer interface of the game controller.
